(19) United States
(12) Patent Application Publication
(10) Pub. No.: US 2011/0294879 A1
(43) Pub. Date: Dec. 1, 2011

(54) METHOD OF TREATMENT OF FRAGILE X SYNDROME, DOWN'S SYNDROME, AUTISM AND RELATED DISORDERS

(75) Inventors: Bernd Jandeleit, Menlo Park, CA (US); Mark A. Gallop, Palo Alto, CA (US); Peter A. Virsik, Portola Valley, CA (US); David J. Wustrow, Saratoga, CA (US)

(73) Assignee: XENOPORT, INC., Santa Clara, CA (US)

(21) Appl. No.: 13/118,125

(22) Filed: May 27, 2011

Related U.S. Application Data
(60) Provisional application No. 61/349,665, filed on May 28, 2010.

(51) Int. Cl. A61K 31/255; A61P 25/00; A61P 25/08; A61P 25/18; A61K 31/265; A61P 25/24

(52) U.S. Cl. 514/512; 514/517

(57) ABSTRACT

Disclosed herein are methods of treating fragile X syndrome, fragile X-associated tremor/ataxia syndrome, Down's syndrome and/or autism, comprising administering a prodrug of acamprosate to a subject suffering therefrom, wherein, in certain embodiments, the acamprosate prodrugs comprise compounds of Formula (I).

### CROSS-REFERENCE TO RELATED APPLICATIONS

This patent application claims priority to U.S. Provisional Application No. 61/349,665, filed May 28, 2010, entitled “Methods of Treatment of Fragile X Syndrome, Down's Syndrome, Autism and Related Disorders”, the contents of which are incorporated herein by reference in their entirety.

### FIELD

Disclosed herein is method of treating a subject, comprising administering a pharmaceutical composition comprising at least one prodrug of acamprosate to a subject having at least one condition selected from fragile X syndrome, fragile X-associated tremor/ataxia syndrome, Down's syndrome and/or autism.

### BACKGROUND

Mental retardation, Down's syndrome, fragile X syndrome and autism are developmental and genetic disorders that affect day to day functioning, including learning, memory, speech, social skills and behavior. Mental retardation means that a subject has lower than average intelligence. Intelligence describes a subject's ability to think, learn and solve problems. A subject with mental retardation may have difficulty learning, may take longer to learn social skills, such as how to communicate, and may be less able to care for himself or herself and to live on his or her own as an adult.

Down's syndrome is a disorder that includes a combination of birth defects, including some degree of mental retardation, characteristic facial features and, often, heart defects, increased infections, problems with vision and hearing, and other health problems. The severity of these problems varies greatly among affected subjects. Down's syndrome is generally is caused by an extra copy chromosome 21 and is also referred to as trisomy 21.

Fragile X syndrome (FXS), as implied by its name, is associated with a fragile site expressed as an isochromatid gap in the metaphase chromosome at map position Xq 27.3. Fragile X syndrome is a genetic disorder caused by a mutation in the 5′-untranslated region of the fragile X mental retardation 1 (FMR1) gene, located on the X chromosome. The mutation that causes fragile X syndrome is associated with a CGG repeat in the fragile X mental retardation gene FMR1. In most healthy individuals, the total number of CGG repeats ranges from less than 10 to 40, with an average of about 29. In fragile X syndrome, the CGG sequence is repeated from 200 to more than 1,000 times. When a subject has more than about 200 CGG sequence repeats, the fragile X gene becomes hypermethylated, which silences the gene. As a result, fragile X mental retardation protein (FMRP) is not produced and the subject is diagnosed as having fragile X syndrome (see, for example, U.S. Pat. Nos. 6,107,025 and 6,180,337).

Premutation expansions (55-200 CGG repeats) of the FMR1 gene are frequent in the general population, with an estimated prevalence of 1 per 259 females and 1 per 812 males (Rousseau et al, Am J. Hum. Genet. 1995, 57: 1006-18; Dombrowski et al, Hum. Mol. Genet. 2002, 11: 371-8). Carriers of the premutation typically have normal IQ, although emotional problems such as anxiety are common. Older male carriers of the premutation (50 years and older) develop progressive intention tremor and ataxia (Hagerman et al, Neurology, 2001, 57: 127-30; Leehey et al, Arch. Neurol. 2003, 60: 117-21). These movement disorders are frequently accompanied by progressive cognitive and behavioral difficulties, including memory loss, anxiety, deficits of executive function, reclusive or irritable behavior, and dementia (Jacquemont et al, JAMA 2004, 291: 460-9). This disorder has been designated fragile X-associated tremor/ataxia syndrome (FXTAS) (Jacquemont et al, Am. J. Hum. Genet. 2003, 72: 869-78). Magnetic resonance imaging in subjects with FXTAS reveals increases in T2-weighted signal intensity in the middle cerebellar peduncles and adjacent cerebellar white matter (Brunberg et al, Am. J. Neuroradiol. 2002, 23: 1757-66).

Fragile X syndrome segregates as an X-linked dominant disorder with reduced penetrance. Either sex when carrying the fragile X mutation may exhibit mental deficiency, which is variable in severity. Children and adults with fragile X syndrome have varying degrees of mental retardation or learning disabilities and behavioral and emotional problems, including autistic-like features and tendencies. Young children with fragile X syndrome often have delays in developmental milestones, such as learning how to sit, walk and talk. Affected children may have frequent tantrums, difficulties in paying attention, frequent seizures (e.g., temporal lobe seizures) are often highly anxious, easily overwhelmed, can have sensory hyperarousal disorder, gastrointestinal disorders, may have speech problems and unusual behaviors, such as hand flapping and hand biting.

Fragile X syndrome can be diagnosed by an established genetic test performed on a sample (e.g., blood sample, buccal sample) from the subject. The test determines whether a mutation or pre-mutation is present in the FMR1 gene of the subject.

Subjects with fragile X syndrome can also have autism, attention deficient disorder and/or obsessive compulsive disorder. Fragile X syndrome is a prevalent form of inherited mental retardation and is characterized by developmental delay, hyperactivity, attention deficit disorder and autistic-like behaviors (Jin, P., et al., Hum Mol Genet. 9: 901-908 (2000)). About 5% of all children diagnosed with autism have a mutation in the FMR1 gene and also have fragile X syndrome (FXS). About 15 to about 20% of subjects with fragile X syndrome meet the full diagnostic criteria for autism. Although mental retardation is a hallmark feature of fragile X syndrome, subjects with fragile X syndrome often display autistic features ranging from shyness, poor eye contact, and social anxiety in mild cases to hand flapping, hand biting and perseverative speech in the severely affected. Subjects with fragile X syndrome display other symptoms associated with autism such as attention deficit and hyperactivity, seizures, hypersensitivity to sensory stimuli, obsessive-compulsive behavior and altered gastrointestinal function. The FMR1 mutation prevents expression of a single protein (FMRP). Brain development in the absence of FMRP gives rise to the major symptoms of fragile X syndrome.

In addition to core symptoms, children with fragile X syndrome frequently have serious behavioral disturbances such as irritability, aggression and self-injurious behaviors. In a recent study of males with fragile X syndrome (ages 8-24), self-injurious behavior was reported in 79%, and aggressive behavior in 75%, of subjects during a two month observation period (Hessl, D., et al., The National Fragile X Foundation Quarterly, Issue 25:10-13 (2006)).

Currently available treatment regimens for humans with mental retardation, Down's syndrome and fragile X syndrome include, for example, behavioral modifications and treatment with a range of medications including antidepressant and antipsychotic drugs. Cognitive behavioral therapy has been used to improve language and socialization in fragile X syndrome and autism. In addition, many classes of psychiatric drugs are used in clinical practice to treat symptoms and behavior in both populations (Berry-Kravis, E. et al., Ment. Retard. Devel Disabil. Res. Rev. 10:42-48 (2004); Malone, R. P., et al., CNS Drugs 19:923-924 (2005)). In recent years, pharmacological treatment with the atypical antipsychotic risperidone has been commonly employed to augment non-pharmacological approaches in the treatment of individuals with autism. A randomized placebo-controlled trial of risperidone in autistic children demonstrated significant improvement on the irritability subscale of the Aberrant Behavior Checklist and the Clinical Global Impressions-Improvement (McCracken, J. T., et al., N. Engl. J. Med. 347:314-321 (2002)). However, adverse events included weight gain, increased appetite, fatigue, drowsiness, dizziness, and drooling. Social isolation and communication were not improved by administration of risperidone and adverse side effects such as extrapyramidal symptoms and dyskinesias have been associated with risperidone use in autistic children (Malone, R. P., et al., J. Am. Acad. Child Adolescent. Psychiatry 41:140-147 (2002)). Since current treatment regimens are frequently not effective or may produce undesirable side-effects with long term use, particularly the use of antipsychotic drugs, there is a need to develop new treatments.

A key tool allowing for a better understanding of the function of FMRP and the identification of new therapies for treatment of fragile X syndrome and related disorders has been development of the Fmr1 knockout (KO) mouse. Initial studies of the behavioral phenotype of the Fmr1 KO mouse on a mixed genetic background reported that the Fmr1 KO mice displayed increased exploratory and locomotor activity compared to wild-type controls, and also a slight learning impairment in the Morris water maze (Bakker, C. E., et al., Cell 78:23-33 (1994)). This learning impairment has been further analyzed by several groups using the Morris water task, plus-shaped water maze, operant conditioning paradigms, conditioned fear, passive avoidance and the radial maze (Bakker, C. E., et al., supra). It is likely that learning and memory performance of Fmr1 KO mice is dependent on genetic background (Paradee, W., et al., Neuroscience 94:185-192 (1999)). Fmr1 KO mice are hyperactive, have altered responses on tests of anxiety, and altered sensorimotor gating (Mineur, Y. S., et al., Hippocampus 12:39-46 (2002)). FMRP can regulate behavioral states of activity/arousal, anxiety-related responses, and social interactions (Bakker, C. E., et al., supra); Peier, A. M., et al., Hum. Mol. Genet. 9:1145-1159 (2000)).

By challenging Fmr1 KO mice with different test situations, the KO mice are hyperactive, can display increased anxiety-like responses, show abnormal social interactions, and have poor learning and memory. Fmr1 KO mice display several abnormal behavioral responses that parallel symptoms of FXS. These behavioral responses depend on the genetic background of the Fmr1 KO mouse. Fmr1 KO mice having particular genetic backgrounds display increased ‘autistic-like’ traits. Specifically, Fmr1 KO mice on a C57BL/6J X DBA/2 F1 (D2-Fmr1 F1) hybrid background display increased stereotypes in the open-field, increased obsessive-like responding in the marble-burying task, and have reduced social interactions, while Fmr1 KO mice on a C57BL/6J X 129S1/SvImJ F1 (129-Fmr1 F1) hybrid background appear to have poor social recognition. That only some of the Fmr1 KO strains display increased ‘autistic-like’ traits is consistent with the observations that only 15-20% of FXS individuals have autism, and may have variation in FXS due to genetic background. Other mouse models of FXS can display unique autistic-like features. (Spencer, C. M., et al., Genes, Brain and Behavior, 4:420-430 (2005)).

Evidence has emerged that FMRP is involved in activity-dependent local synaptic protein synthesis. The major excitatory neurotransmitter glutamate, via group I metabotropic glutamate receptors (mGluRs), stimulates protein synthesis in dendrites. The Group I mGluRs are a subgroup of the G-protein coupled mGluR family, and are composed of two subtypes, mGluR1 and mGluR5. FMR1 mRNA is present in dendrites and FMRP is synthesized in response to mGluR activation of synaptoneurosomes (Weiler, et al., Proc. Natl. Acad. Sci. USA 90, 7168-7171 (1993)). Because FMRP itself can regulate mRNA translation, the synthesis of FMRP at synapses in response of mGluR activation may be a mechanism by which neuronal activity can regulate or control synthesis of other proteins important for synaptic plasticity and development.

Several studies have demonstrated that activation of group I mGluRs with either synaptic stimulation or the selective agonist R,S-dihydroxyphenylglycine (DHPG) induces long-term depression (LTD) of synaptic responses in area CA1 of the rat hippocampus (Fitzjohn et al., Neuropharmacology 38: 1577-1584, 1999; Kemp and Bashir, Neuropharmacology 38: 495-504, 1999; Huber et al., Science 288: 1254-1257, 2000). LTD is dependent on mGluR5 and most importantly requires the rapid dendritic synthesis of new proteins. It has been suggested that an LTD-like mechanism could be responsible for elimination or pruning of inappropriate synapses which are formed during early periods of postnatal development and there is evidence that activation of mGluR5 results in decreases in synaptic strength. A role for FMRP in LTD was discovered using the Fmr1 knockout mouse model of fragile X syndrome indicating that FMRP may normally function as an inhibitor of mGluR-dependent protein synthesis and, in the absence of FMRP, there is unregulated synthesis of the proteins required for LTD. One implication of these results is that an excess of LTD or a synapse elimination mechanism in Fmr1 knockout mice or fragile X subjects may perturb the normal synaptic development process and lead to abnormalities in dendritic spine structure and eventually to cognitive deficits. Alternatively or in addition, the enhancement of an LTD-like mechanism in the adult could result in the ineffective storage of information in the brain which could also contribute to mental retardation.

The pathophysiological impact of unregulated mGluR-dependent protein synthesis in FXS has been termed the “mGluR theory of fragile X” (Bear et al, Trends Neurosci. 27: 370-377 (2004)) and has prompted investigations of blockade of signaling through group I mGluR's as methods for treating fragile X syndrome and related disorders using genetic or pharmacological tools (Dolen et al, Pharmacology & Therapeutics (2010)). In particular, small molecule antagonists of mGluR5 (e.g. 2-methyl-6-(phenylethynyl)-pyridine, MPEP) have been found to reverse behavioral and developmental phenotypes in Fmr KO mice (Bear et al, U.S. Pat. No. 6,890,931; U.S. Pat. No. 6,916,821; U.S. Pat. No. 7,648,993) and data from clinical studies in fragile X of fenobam and AFQ056 support the efficacy of mGluR5 antagonists in humans (Berry-Kravis et al, J. Med. Genet. 46: 266-271 (2009)).

3-(Acetylamino)propylsulfonic acid, also referred to as N-acetylhomotaurine or acamprosate:

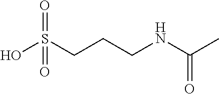

is a derivative of homotaurine, a naturally occurring structural analog of γ-aminobutyric acid (GABA) that appears to affect multiple receptors in the central nervous system (CNS). As an antiglutamatergic agent, acamprosate is believed to exert a neuropharmacological effect as an antagonist of N-methyl-D-aspartate (NMDA) receptors. The mechanism of action is believed to include blocking of the Ca²⁺ channel to slow Ca²⁺ influx and reduce the expression of c-fos, leading to changes in messenger RNA transcription and the concomitant modification to the subunit composition of NMDA receptors in selected brain regions (Zornoza et al., CNS Drug Reviews, 2003, 9(4), 359-374; and Rammes et al., Neuropharmacology 2001, 40, 749-760). There is also evidence that acamprosate may interact with excitatory glutamatergic neurotransmission in general and as an antagonist of the metabotropic glutamate receptor subtype 5 (mGluR5) in particular (De Witte et al., CNS Drugs 2005, 19(6), 517-37). The glutamatergic mechanism of action of acamprosate may explain the effects of acamprosate on alcohol dependence and suggests other therapeutic activities such as in neuroprotection.

Consistent with its activity as a functional glutamaterigic antagonist, a clinical study of CAMPRAL® (acamprosate calcium) in subjects with FXS and comorbid autistic disorder demonstrated significant symptomatic improvements as assessed by the investigators' Clinical Global Impression (CGI-I), with improvements in communication and social interaction skills being particularly noteworthy (Erickson et al, J. Autism Dev. Disord. 2010). Gastrointestinal distress (nausea and vomiting) are commonly observed side effects in subjects receiving acamprosate, and adverse GI events were observed in the majority of fragile X subjects treated in this study.

Acamprosate is a polar molecule that lacks the requisite physicochemical characteristics for effective passive permeability across cellular membranes. As a consequence, the oral bioavailability of acamprosate in humans is only about 11%, and poor absorption of the drug from the GI tract likely contributes to its limited tolerability.

### SUMMARY

Disclosed herein are acamprosate prodrugs exhibiting enhanced absorption from the lower gastrointestinal tract, having the potential to increase the oral bioavailability of the drug, to facilitate administration of acamprosate using sustained release oral dosage forms, and to have improved tolerabilty in the treatment of fragile X syndrome, autism and related disorders.

The present disclosure provides methods of treating disorders, including but not limited to Fragile X syndrome, fragile X-associated tremor/ataxia syndrome, Down's syndrome and other forms of mental retardation, and autism, comprising administering to a subject (e.g., a human) an acamprosate prodrug. In certain embodiments, the acamprosate prodrug is selected from an acamprosate prodrug disclosed in one of the following US Patent Publications: Li et al., US 2009/0099253 published Apr. 16, 2009; Jandeleit et al., US 2009/0069419 published Mar. 12, 2009; Jandeleit et al., US 2009/0082464 published Mar. 26, 2009; Jandeleit et al., US 2009/0082440 published Mar. 26, 2009; and Jandeleit et al., US 2009/0076147 published Mar. 19, 2009. In certain embodiments, the acamprosate prodrugs are those disclosed in Jandeleit et al., US 2009/0076147, which comprise compounds of Formula (I):

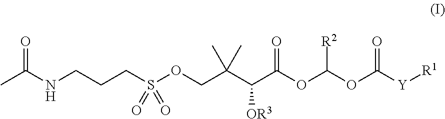

or a pharmaceutically acceptable salt thereof, wherein:

Y is chosen from —O— and a bond;

R¹ is chosen from C₁₋₆ alkyl, substituted C₁₋₆ alkyl, C₃₋₆ cycloalkyl, substituted C₃₋₆ cycloalkyl, phenyl, substituted phenyl, C₄₋₁₂ cycloalkylalkyl, substituted C₄₋₁₂ cycloalkylalkyl, C₇₋₁₂ arylalkyl, substituted C₇₋₁₂ arylalkyl, C₁₋₆ heteroalkyl, substituted C₁₋₆ heteroalkyl, C₃₋₆ heterocycloalkyl, substituted C₃₋₆ heterocycloalkyl, C₅₋₆ heteroaryl, substituted C₅₋₆ heteroaryl, C₄₋₁₂ heterocycloalkylalkyl, substituted C₄₋₁₂ heterocycloalkylalkyl, C₆₋₁₂ heteroarylalkyl, substituted C₆₋₁₂ heteroarylalkyl, and —(CHR⁶)ₙ—OPO(OH)₂ wherein n is chosen from 1, 2, and 3, and each R⁶ is independently chosen from hydrogen and methyl;

R² is chosen from hydrogen, C₁₋₆ alkyl, substituted C₁₋₆ alkyl, C₃₋₆ cycloalkyl, substituted C₃₋₆ cycloalkyl, phenyl, and substituted phenyl; and

R³ is chosen from hydrogen, —PO(OH)₂, and —C(O)R⁴ wherein R⁴ is C₁₋₄ alkyl.

In certain embodiments of a compound of Formula (I), each substituent group is independently chosen from halogen, —OH, C₁₋₄ alkyl, C₁₋₄ alkoxy, and —N(R⁵)₂ wherein each R⁵ is independently chosen from hydrogen and C₁₋₂ alkyl. In certain embodiments of a compound of Formula (I), each substituent group is independently chosen from halogen, —OH, and C₁₋₄ alkyl. In certain embodiments of a compound of Formula (I), each substituent group is independently chosen from —OH, C₁₋₄ alkyl, and C₁₋₄ alkoxy.

The present disclosure provides: a) the use of a compound of Formula (I) for the treatment of Down's syndrome, Fragile X syndrome, fragile X-associated tremor/ataxia syndrome and other forms of mental retardation, and autism; b) the use of a compound of Formula (I) in the manufacture of a pharmaceutical composition for the treatment of Down's syndrome, Fragile X syndrome and other forms of mental retardation, and autism; c) methods of treating Down's syndrome, Fragile X syndrome and other forms of mental retardation, and autism in a subject in need of such treatment, comprising administering to such subject a therapeutically effective amount of a compound of Formula (I); and d) methods of treating Down's syndrome, Fragile X syndrome and other forms of mental retardation, and autism in a subject in need of such treatment, comprising administering to such subject a therapeutically effective amount of a pharmaceutical composition comprising a compound of Formula (I). In certain embodiments, the composition is for oral administration.

Certain embodiments relate to methods for treating Down's syndrome, Fragile X syndrome and other forms of mental retardation, and autism, comprising co-administering other therapeutic agents (e.g., simultaneously or at different times) to the subject (human or other animal) with an amount of a compound of Formula (I) sufficient to treat the disorder.

In another aspect, the disclosure relates to methods for preparing a pharmaceutical composition, comprising combining a compound of Formula (I) together with a suitable amount of one or more pharmaceutically acceptable vehicles so as to provide a composition for administration to a subject.

In other embodiments, the method comprises administering to the subject an effective amount of one or more compounds of Formula (I), either alone or in combinations thereof. In certain aspects, a compound of Formula (I) is administered in a dose ranging from about 1 to about 200 mg/kg body weight/day. In some embodiments, a compound of Formula (I) is administered in a dose ranging from about 5 to about 100 mg/kg body weight/day.

In other embodiments, the disclosure is directed to methods of treating anxiety in a subject having Fragile X syndrome, comprising administering to the subject a compound of Formula (I).

In other embodiments, the disclosure is directed to methods of treating epilepsy in a subject having Fragile X syndrome, comprising administering to the subject a compound of Formula (I).

In other embodiments, the disclosure is directed to methods of treating anxiety in a subject having a disorder selected from autism, mental retardation and Down's syndrome, comprising administering to the subject a compound of Formula (I).

In other embodiments, the disclosure is directed to methods of treating epilepsy in a subject having a disorder selected from autism, mental retardation and Down's syndrome, comprising administering to the subject a compound of Formula (I).

In other embodiments, the disclosure is directed to methods of treating a subject having autism, comprising administering a compound of Formula (I) to the subject.

In other embodiments, the disclosure is directed to methods of treating a subject having autism and fragile X syndrome comprising administering an effective amount of a compound of Formula (I) to the subject.

Treatment of subjects with a compound of Formula (I) can halt, diminish, inhibit, reverse or ameliorate conditions associated with mental retardation (e.g., anxiety, epilepsy, autism and Fragile X), thereby increasing the quality of life for subjects afflicted with mental retardation conditions.

### DETAILED DESCRIPTION

Definitions

A dash (“-”) that is not between two letters or symbols is used to indicate a point of bonding to a moiety or substituent. For example, —CONH₂ is attached through the carbon atom.

“Alkyl” by itself or as part of another substituent refers to a saturated or unsaturated, branched or straight-chain, monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like. The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group can have from 1 to 20 carbon atoms, in certain embodiments, from 1 to 10 carbon atoms, in certain embodiments from 1 to 8 carbon atoms, in certain embodiments, from 1 to 6 carbon atoms, in certain embodiments from 1 to 4 carbon atoms, and in certain embodiments, from 1 to 3 carbon atoms.

“Alkoxy” by itself or as part of another substituent refers to a radical —OR³¹ where R³¹ is chosen from alkyl, heteroalkyl, cycloalkyl, heterocycloalkyl, cycloalkylalkyl, heterocycloalkylalkyl, aryl, heteroaryl, arylalkyl, and heteroarylalkyl, as defined herein. Examples of alkoxy groups include, but are not limited to, methoxy, ethoxy, propoxy, butoxy, cyclohexyloxy, and the like. In certain embodiments, an alkoxy group is C₁₋₁₈ alkoxy, in certain embodiments, C₁₋₁₂ alkoxy, in certain embodiments, C₁₋₈ alkoxy, in certain embodiments, C₁₋₆ alkoxy, in certain embodiments, C₁₋₄ alkoxy, and in certain embodiments, C₁₋₃ alkoxy.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 6 to 20 carbon atoms (C₆₋₂₀), from 6 to 12 carbon atoms (C₆₋₁₂), and in certain embodiments, from 6 to 10 carbon atoms (C₆₋₁₀).

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl or alkynyl moiety of the arylalkyl group is C₁₋₁₀ and the aryl moiety is C₇₋₂₀, in certain embodiments, an arylalkyl group is C₆₋₁₈ arylalkyl, e.g., the alkanyl, alkenyl or alkynyl moiety of the arylalkyl group is C₁₋₈ and the aryl moiety is C₆₋₁₀.

“Autism” is a developmental neurological disorder that affects the normal functioning of the brain. The disorder may be characterized by the degree to which a subject has certain behavioral symptoms, including deficits in sociability, reciprocal verbal and nonverbal communication, restricted, repetitive or stereotypical behavior, difficulties in verbal and non-verbal communication, social interactions, and leisure or play activities. In certain instances, autism may result from abnormalities related to neurotransmitters including serotonin, norepinephrine, and histamine. Causative factors may include rubella, problems during pregnancy, labor and delivery, cytomegalic inclusion disease, phenylketonuria, Fragile X syndrome, and genetic predisposition for autism.

“Bioavailability” refers to the rate and amount of a drug that reaches the systemic circulation of a subject following administration of the drug or prodrug thereof to the subject and can be determined by evaluating, for example, the plasma or blood concentration-versus-time profile for a drug.

“Compounds” of Formula (I) disclosed herein include any specific compounds within this formula. Compounds may be identified either by their chemical structure and/or chemical name. When the chemical structure and chemical name conflict, the chemical structure is determinative of the identity of the compound. The compounds described herein may comprise one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to those skilled in the art.

Compounds of Formula (I) include optical isomers of compounds of Formula (I), racemates thereof, and other mixtures thereof. In such embodiments, the single enantiomers or diastereomers, i.e., optically active forms, can be obtained by asymmetric synthesis or by resolution of the racemates. Resolution of the racemates may be accomplished, for example, by conventional methods such as crystallization in the presence of a resolving agent, or chromatography, using, for example, a chiral high-pressure liquid chromatography (HPLC) column. In addition, compounds of Formula (I) include Z- and E-forms (or cis- and trans-forms) of compounds with double bonds.

Compounds of Formula (I) may also exist in several tautomeric forms including the enol form, the keto form, and mixtures thereof. Accordingly, the chemical structures depicted herein encompass all possible tautomeric forms of the illustrated compounds. Compounds of Formula (I) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds as referred to herein may be free acid, salts, hydrated, solvated, or N-oxides. Thus, when reference is made to compounds of the present disclosure, such as compounds of Formula (I), it is understood that a compound also implicitly refers to free acid, salts, solvates, hydrates, N-oxides, and combinations of any of the foregoing. Certain compounds may exist in multiple crystalline, cocrystalline, or amorphous forms. Compounds of Formula (I) include pharmaceutically acceptable solvates of the free acid or salt form of any of the foregoing, hydrates of the free acid or salt form of any of the foregoing, as well as crystalline forms of any of the foregoing.

Compounds of Formula (I) may be solvates. The term “solvate” refers to a molecular complex of a compound with one or more solvent molecules in a stoichiometric or non-stoichiometric amount. Such solvent molecules are those commonly used in the pharmaceutical art, which are known to be innocuous to a subject, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

“Cycloalkyl” by itself or as part of another substituent refers to a saturated or partially unsaturated cyclic alkyl radical. Where a specific level of saturation is intended, the nomenclature “cycloalkanyl” or “cycloalkenyl” is used. Examples of cycloalkyl groups include groups derived from cyclopropane, cyclobutane, cyclopentane, cyclohexane, and the like. In certain embodiments, a cycloalkyl group is C₃₋₁₅ cycloalkyl, C₃₋₁₂ cycloalkyl, C₃₋₁₀ cycloalkyl or in certain embodiments, C₃₋₈ cycloalkyl. Cycloalkyl includes nonaromatic fused ring systems.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₇₋₃₀ cycloalkylalkyl, e.g., the alkanyl, alkenyl, or alkynyl moiety of the cycloalkylalkyl group is C₁₋₁₀ and the cycloalkyl moiety is C₆₋₂₀, and in certain embodiments, a cycloalkylalkyl group is C₇₋₂₀ cycloalkylalkyl, e.g., the alkanyl, alkenyl, or alkynyl moiety of the cycloalkylalkyl group is C₁₋₈ and the cycloalkyl moiety is C₄₋₂₀ or C₆₋₁₂. In certain embodiments, a cycloalkylalkyl group is C₄₋₁₈ cycloalkylalkyl.

The “(1S)-diastereomer” of a compound of Formula (I) refers to a compound in which the stereochemical configuration of the acetal carbon is (S). The “(1R)-diastereomer” of a compound of Formula (I) refers to a compound in which the stereochemical configuration of the acetal carbon is (R).

“Disease” refers to a disease, disorder, condition, or symptom of any of the foregoing.

“Down's syndrome” refers to a chromosomal dysgenesis of one or more abnormalities caused by triplication of chromosome 21 (trisomy 21), partial triplication of chromosome 21, or translocation of chromosome 21. Abnormalities and phenotypic disorders include mental retardation, retarded growth, flat hypoplastic face with short nose and prominent epicanthic skin folds, small low-set ears with prominent antihelix, fissured and thickened tongue, laxness of joint ligaments, pelvic dysplasia, broad hands and feet, stubby fingers, transverse palmar crease, increased incidence of leukemia and Alzheimers disease, heart and intestinal defects, problems with the immune and endocrine systems, and tissue and skeletal deformities.

Over 90 percent of the individuals affected with Down's syndrome have an extra number 21 chromosome in all of their cells, giving each cell a total of 47 chromosomes rather than the normal 46. For this reason, the condition is also known as “Trisomy 21”. Trisomy 21 results from nondisjunction or failure of chromosomes to separate sometime during either division of meiosis or mitosis. Most Down's syndrome individuals have trisomy 21, and conversely, in individuals who carry a translocation involving chromosome 21, and in mosaics who have both trisomic and normal cells, the characteristics of the syndrome are seen. There are, however, rare forms of Down syndrome in which only part of chromosome 21 is present in triplicate.

“Drug” as defined under 21 U.S.C. §321(g)(1) means “(A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals.”

“Halogen” refers to a fluoro, chloro, bromo, or iodo group. In certain embodiments, halogen is fluoro, and in certain embodiments, halogen is chloro.

“Heteroalkyl” by itself or as part of another substituent refers to an alkyl group in which one or more of the carbon atoms (and certain associated hydrogen atoms) are independently replaced with the same or different heteroatomic groups. Examples of heteroatomic groups include, but are not limited to, —O—, —S—, —O—O—, —S—S—, —O—S—, NR³⁷, ═N—N═, —N═N—, —N═N—NR³⁷—, —PR³⁷—, —P(O)₂—, —POR³⁷—, —O—P(O)₂—, —SO—, —SO₂—, —Sn(R³⁷)₂—, and the like, where each R³⁷ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₅₋₁₂ heteroaryl, substituted C₅₋₁₂ heteroaryl, C₆₋₁₈ heteroarylalkyl, or substituted C₆₋₁₈ heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatom, groups having four carbon atoms and two heteroatoms, etc. In certain embodiments, each R³⁷ is independently chosen from hydrogen and C₁₋₃ alkyl. In certain embodiments, a heteroatomic group is chosen from —O—, —S—, —NH—, —N(CH₃)—, and —SO₂—.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. Heteroaryl encompasses 5- to 7-membered aromatic, monocyclic rings containing one or more, for example, from 1 to 4, or in certain embodiments, from 1 to 3, heteroatoms chosen from N, O, and S, with the remaining ring atoms being carbon; and 5- to 14-membered bicyclic rings containing one or more, for example, from 1 to 4, or in certain embodiments, from 1 to 3, heteroatoms chosen from N, O, and S, with the remaining ring atoms being carbon, wherein at least one of the rings is an aromatic ring, and wherein at least one heteroatom is present in the at least one aromatic ring. For example, heteroaryl includes a 5- to 7-membered heteroaromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. In certain embodiments, a heteroaryl group is C₅₋₁₂ heteroaryl, C₅₋₁₀ heteroaryl, and in certain embodiments, C₅₋₆ heteroaryl. The ring of a C₅₋₁₀ heteroaryl has from 4 to 9 carbon atoms, with the remainder of the atoms in the ring being heteroatoms.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, in certain embodiments from 5- to 10-membered heteroaryl, and in certain embodiments from 5- to 8-heteroaryl. In certain embodiments heteroaryl groups are those derived from thiophene, pyrrole, benzothiophene, benzofuran, indole, pyridine, quinoline, imidazole, oxazole, or pyrazine.

“Heteroarylalkyl” by itself or as part of another substituent refers to an acyclic alkyl radical in which one of the hydrogen atoms bonded to a carbon atom, is replaced with a heteroaryl group. Typically a terminal or sp³ carbon atom is the atom replaced with the heteroaryl group. Where specific alkyl moieties are intended, the nomenclature “heteroarylalkanyl,” “heteroarylalkenyl,” and “heterorylalkynyl” is used. In certain embodiments, a heteroarylalkyl group is a 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl, and in certain embodiments, 6- to 14-membered heteroarylalkyl, e.g., the alkanyl, alkenyl, or alkynyl moiety of the heteroarylalkyl is 1- to 4-membered and the heteroaryl moiety is a 5- to 12-membered heteroaryl. In certain embodiments, a heteroarylalkyl group is C₆₋₁₈ heteroarylalkyl and in certain embodiments, C₆₋₁₀ heteroarylalkyl.

“Heterocycloalkyl” by itself or as part of another substituent refers to a saturated or partially unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “heterocycloalkanyl” or “heterocycloalkenyl” is used. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like. Heterocycloalkyl includes nonaromatic heterocycloalkyl fused ring systems. In certain embodiments, a heterocycloalkyl group is a C₃₋₁₂ heterocycloalkylalkyl, C₃₋₁₀ heterocycloalkylalkyl, and in certain embodiments C₃₋₈ heterocycloalkyalkyl.

“Heterocycloalkyalkyl” by itself or as part of another substituent refers to an acyclic alkyl radical in which one of the hydrogen atoms bonded to a carbon atom, is replaced with a heterocycloalkyl group as defined herein. In certain embodiments, a heterocycloalkylalkyl group is a C₄₋₁₈ heterocycloalkylalkyl, C₄₋₁₂ heterocycloalkylalkyl, and in certain embodiments C₄₋₁₀ heterocycloalkyalkyl.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to an aromatic ring system in which one or more carbon atoms (and any associated hydrogen atoms) are independently replaced with the same or different heteroatom in such a way as to maintain the continuous π (pi)-electron system characteristic of aromatic systems and a number of out-of-plane π (pi)-electrons corresponding to the Hückel rule (4n+2). Examples of heteroatoms to replace the carbon atoms include, but are not limited to, N, P, O, S, Si, etc. In certain embodiments, a heteroatom is chosen from N, O, and S. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, tromethamine, and the like. In certain embodiments, pharmaceutically acceptable addition salts include metal salts such as sodium, potassium, aluminum, calcium, magnesium and zinc salts, and ammonium salts such as tromethamine, isopropylamine, diethylamine, and diethanolamine salts. In certain embodiments, a pharmaceutically acceptable salt is the hydrochloride salt. In certain embodiments, a pharmaceutically acceptable salt is the sodium salt. Pharmaceutically acceptable salts may be prepared by the skilled chemist, by treating, for example, a compound of Formula (I) with an appropriate base in a suitable solvent, followed by crystallization and filtration.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a subject and which does not destroy the pharmacological activity thereof and which is non-toxic when administered in doses sufficient to provide a therapeutically effective amount of the compound.

“Pharmaceutical composition” refers to at least one compound of Formula (I) and at least one pharmaceutically acceptable vehicle with which the at least one compound of Formula (I) is administered to a subject.

“Prodrug” refers to a derivative of a drug molecule that requires a transformation within the body to release the active drug. Prodrugs are frequently, although not necessarily, pharmacologically inactive until converted to the parent drug. Prodrugs may be obtained by bonding a promoiety typically via a functional group, to a drug. For example, referring to compounds of Formula (I), the promoiety is bonded to the drug, acamprosate, via the sulfonic acid functional group of acamprosate. Compounds of Formula (I) are prodrugs of acamprosate that can be metabolized within a subject's body to release acamprosate.

“Salt” refers to a chemical compound consisting of an assembly of cations and anions. Salts of a compound of the present disclosure include stoichiometric and non-stoichiometric forms of the salt. In certain embodiments, because of its potential use in medicine, salts of a compound of Formula (I) are pharmaceutically acceptable salts.

“Subject” refers to a mammal, for example, a human.

“Substituted” refers to a group in which one or more hydrogen atoms are independently replaced with the same or different substituent group(s). Examples of substituent groups include, but are not limited to, -M, —R⁶⁰, —O⁻, ═OR⁶⁰, —SR⁶⁰, —S⁻, ═S, —NR⁶⁰R⁶¹, ═NR⁶⁰, CF₃, —CN, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, and —C(NR⁶²)NR⁶⁰R⁶¹ where M is halogen; R⁶⁰, R⁶¹, R⁶², an d R⁶³ are independently chosen from hydrogen, alkyl, alkoxy, cycloalkyl, heterocycloalkyl, aryl, and heteroaryl, or R⁶⁰ and R⁶¹ together with the nitrogen atom to which they are bonded form a ring chosen from a heterocycloalkyl ring. In certain embodiments, R⁶⁰, R⁶¹, R⁶², and R⁶³ are independently chosen from hydrogen, C₁₋₆ alkyl, C₁₋₆ alkoxy, C₃₋₁₂ cycloalkyl, C₃₋₁₂ heterocycloalkyl, C₆₋₁₂ aryl, and C₆₋₁₂ heteroaryl. In certain embodiments, each substituent group is independently chosen from halogen, —OH, —CN, —CF₃, ═O, NO₂, C₁₋₃ alkoxy, C₁₋₃ alkyl, —COOR⁶⁴ wherein R⁶⁴ is chosen from hydrogen and C₁₋₃ alkyl, and —N(R⁶⁵)₂ wherein each R⁶⁵ is independently chosen from hydrogen and C₁₋₃ alkyl. In certain embodiments, each substituent group is independently chosen from halogen, —OH, —CN, —CF₃, —OCF₃, ═O, NO₂, C₁₋₆ alkoxy, C₁₋₆ alkyl, —COOR²⁶, —N(R²⁷)₂, and —CON(R²⁸)₂; wherein each of R²⁶, R²⁷, and R²⁸ is independently chosen from hydrogen and C₁₋₆ alkyl.

In certain embodiments, each substituent group is independently chosen from halogen, —OH, —CN, —CF₃, ═O, —NO₂, C₁₋₃ alkoxy, C₁₋₃ alkyl, —COOR¹² wherein R¹² is chosen from hydrogen and C₁₋₃ alkyl, and —N(R¹²)₂ wherein each R¹² is independently chosen from hydrogen and C₁₋₃ alkyl. In certain embodiments, each substituent group is independently chosen from halogen, —OH, —CN, —CF₃, —OCF₃, ═O, —NO₂, C₁₋₆ alkoxy, C₁₋₆ alkyl, —COOR¹², —N(R¹²)₂, and —CONR¹²₂; wherein each R¹² is independently chosen from hydrogen and C₁₋₆ alkyl. In certain embodiments, each substituent group is chosen from C₁₋₄ alkyl, —OH, and —NH₂.

“Sustained release” refers to release of a compound from a dosage form of a pharmaceutical composition at a rate effective to achieve a therapeutic or prophylactic concentration of the compound or active metabolite thereof, in the systemic circulation of a subject over a prolonged period of time relative to that achieved by administration of an immediate release formulation of the same compound by the same route of administration. In some embodiments, release of a compound occurs over a time period of at least about 4 hours, such as at least about 8 hours, at least about 12 hours, at least about 16 hours, at least about 20 hours, and in some embodiments, at least about 24 hours.

“Treating” or “treatment” of any disease refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the subject. In certain embodiments, “treating” or “treatment” refers to delaying the onset of the disease or at least one or more symptoms thereof in a subject which may be exposed to or predisposed to a disease or disorder even though that subject does not yet experience or display symptoms of the disease.

“Therapeutically effective amount” refers to the amount of a compound that, when administered to a subject for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the subject to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a subject. A therapeutically effective dose may vary from compound to compound, and from subject to subject, and may depend upon factors such as the condition of the subject and the route of delivery. A therapeutically effective dose may be determined in accordance with routine pharmacological procedures known to those skilled in the art.

Reference is now made in detail to certain embodiments of compounds, compositions, and methods. The disclosed embodiments are not intended to be limiting of the claims. To the contrary, the claims are intended to cover all alternatives, modifications, and equivalents.

Compounds

In certain embodiments, methods of treating disorders, including but not limited to Fragile X syndrome, fragile X-associated tremor/ataxia syndrome, Down's syndrome and other forms of mental retardation, and autism is provided, comprising administering to a subject (e.g., a human) an acamprosate prodrug according to the present disclosure.

In certain embodiments, the acamprosate prodrug is a selected from an acamprosate prodrug disclosed in one of the following US Patent Publications: Li et al., US 2009/0099253 published Apr. 16, 2009; Jandeleit et al., US 2009/0069419 published Mar. 12, 2009; Jandeleit et al., US 2009/0082464 published Mar. 26, 2009; and Jandeleit et al., US 2009/0082440 published Mar. 26, 2009.

In other embodiments, the acamprosate prodrug is one of those disclosed in Jandeleit et al., US 2009/0076147 published Mar. 19, 2009 having a chemical structure according to Formula (I):

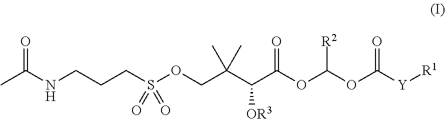

or a pharmaceutically acceptable salt thereof, wherein:

Y is chosen from —O— and a bond;

R¹ is chosen from C₁₋₆ alkyl, substituted C₁₋₆ alkyl, C₃₋₆ cycloalkyl, substituted C₃₋₆ cycloalkyl, phenyl, substituted phenyl, C₄₋₁₂ cycloalkylalkyl, substituted C₄₋₁₂ cycloalkylalkyl, C₇₋₁₂ arylalkyl, substituted C₇₋₁₂ arylalkyl, C₁₋₆ heteroalkyl, substituted C₁₋₆ heteroalkyl, C₃₋₆ heterocycloalkyl, substituted C₃₋₆ heterocycloalkyl, C₅₋₆ heteroaryl, substituted C₅₋₆ heteroaryl, C₄₋₁₂ heterocycloalkylalkyl, substituted C₄₋₁₂ heterocycloalkylalkyl, C₆₋₁₂ heteroarylalkyl, substituted C₆₋₁₂ heteroarylalkyl, and —(CHR⁶)ₙ—OPO(OH)₂ wherein n is chosen from 1, 2, and 3, and each R⁶ is independently chosen from hydrogen and methyl;

R² is chosen from hydrogen, C₁₋₆ alkyl, substituted C₁₋₆ alkyl, C₃₋₆ cycloalkyl, substituted C₃₋₆ cycloalkyl, phenyl, and substituted phenyl; and

R³ is chosen from hydrogen, —PO(OH)₂, and —C(O)R⁴ wherein R⁴ is C₁₋₄ alkyl.

In certain embodiments of a compound of Formula (I), each substituent group is independently chosen from halogen, —OH, C₁₋₄ alkyl, C₁₋₄ alkoxy, and —N(R⁵)₂ wherein each R⁵ is independently chosen from hydrogen and C₁₋₂ alkyl. In certain embodiments of a compound of Formula (I), each substituent group is independently chosen from halogen, —OH, and C₁₋₄ alkyl. In certain embodiments of a compound of Formula (I), each substituent group is independently chosen from —OH, C₁₋₄ alkyl, and C₁₋₄ alkoxy.

In certain embodiments of a compound of Formula (I), Y is —O—; and R¹ is chosen from C₁₋₄ alkyl, substituted C₁₋₄ alkyl, C₅₋₆ cycloalkyl, substituted C₅₋₆ cycloalkyl, phenyl, substituted phenyl, C₆₋₁₀cycloalkylalkyl, substituted C₆₋₁₀ cycloalkylalkyl, C₇₋₁₀ arylalkyl, substituted C₇₋₁₀ arylalkyl, C₁₋₄ heteroalkyl, substituted C₁₋₄ heteroalkyl, C₅₋₆ heterocycloalkyl, substituted C₅₋₆ heterocycloalkyl, C₅₋₆ heteroaryl, substituted C₅₋₆ heteroaryl, C₆₋₁₀ heterocycloalkylalkyl, substituted C₆₋₁₀ heterocycloalkylalkyl, C₆₋₁₀ heteroarylalkyl, substituted C₆₋₁₀ heteroarylalkyl, and —(CHR⁶)ₙ—OPO(OH)₂, wherein n is chosen from 1, 2 and 3, and each R⁶ is independently chosen from hydrogen and methyl.

In certain embodiments of a compound of Formula (I), Y is a bond; and R¹ is chosen from C₁₋₄ alkyl, substituted C₁₋₄ alkyl, C₅₋₆ cycloalkyl, substituted C₅₋₆ cycloalkyl, phenyl, substituted phenyl, C₆₋₁₀ cycloalkylalkyl, substituted C₆₋₁₀ cycloalkylalkyl, C₇₋₁₀ arylalkyl, substituted C₇₋₁₀ arylalkyl, C₁₋₄ heteroalkyl, substituted C₁₋₄ heteroalkyl, C₅₋₆ heterocycloalkyl, substituted C₅₋₆ heterocycloalkyl, C₅₋₆ heteroaryl, substituted C₅₋₆ heteroaryl, C₆₋₁₀ heterocycloalkylalkyl, substituted C₆₋₁₀ heterocycloalkylalkyl, C₆₋₁₀ heteroarylalkyl, substituted C₆₋₁₀ heteroarylalkyl, and —(CHR⁶)ₙ—OPO(OH)₂, wherein n is chosen from 1, 2 and 3, and each R⁶ is independently chosen from hydrogen and methyl.

In certain embodiments of a compound of Formula (I), R¹ is chosen from C₁₋₄ alkyl, phenyl, substituted phenyl, benzyl, substituted benzyl, cyclohexyl, substituted cyclohexyl, and —(CHR⁶)ₙ—OPO(OH)₂, wherein n is chosen from 1, 2 and 3, and each R⁶ is independently chosen from hydrogen and methyl.

In certain embodiments of a compound of Formula (I), R¹ is chosen from methyl, ethyl, n-propyl, isopropyl, n-butyl, isobutyl, sec-butyl, tert-butyl, phenyl, o-tolyl, benzyl, cyclohexyl, and —(CHR⁶)ₙ—OPO(OH)₂, wherein n is chosen from 1, 2 and 3, and each R⁶ is independently chosen from hydrogen and methyl.

In certain embodiments of a compound of Formula (I), R² is chosen from hydrogen, C₁₋₄ alkyl, phenyl, and cyclohexyl. In certain embodiments of a compound of Formula (I), R² is chosen from hydrogen, methyl, ethyl, n-propyl, and isopropyl. In certain embodiments of a compound of Formula (I), R² is chosen from methyl, ethyl, n-propyl, and isopropyl. In certain embodiments of a compound of Formula (I) wherein R² is chosen from methyl, ethyl, n-propyl, and isopropyl; the stereochemistry of the carbon atom to which R² is bonded is of the S-configuration. In certain embodiments of a compound of Formula (I) wherein R² is chosen from methyl, ethyl, n-propyl, and isopropyl; the stereochemistry of the carbon atom to which R² is bonded is of the R-configuration. In certain embodiments of a compound of Formula (I) wherein R² is chosen from methyl, ethyl, n-propyl, and isopropyl; R³ is hydrogen, and in certain embodiments, R³ is —PO(OH)₂.

In certain embodiments of a compound of Formula (I), R³ is chosen from hydrogen and —C(O)R⁴ wherein R⁴ is C₁₋₄ alkyl. In certain embodiments of a compound of Formula (I), R³ is hydrogen; and in certain embodiments R³ is —C(O)R⁴ wherein R⁴ is C₁₋₄ alkyl.

In certain embodiments of a compound of Formula (I), Y is —O—; R¹ is chosen from C₁₋₄ alkyl, phenyl, substituted phenyl, benzyl, substituted benzyl, cyclohexyl, substituted cyclohexyl, and —(CHR⁶)ₙ—OPO(OH)₂, wherein n is chosen from 1, 2 and 3, and each R⁶ is independently chosen from hydrogen and methyl; R² is chosen from hydrogen, C₁₋₄ alkyl, phenyl, and cyclohexyl; and R³ is chosen from hydrogen and —PO(OH)₂. In certain embodiments of a compound of Formula (I), Y is a bond; R¹ is chosen from C₁₋₄ alkyl, phenyl, substituted phenyl, benzyl, substituted benzyl, cyclohexyl, substituted cyclohexyl, and —(CHR⁶)ₙ—OPO(OH)₂, wherein n is chosen from 1, 2 and 3; R² is chosen from hydrogen, C₁₋₄ alkyl, phenyl, and cyclohexyl; and R³ is chosen from hydrogen and —PO(OH)₂.

In certain embodiments of a compound of Formula (I), Y is —O—; R¹ is chosen from methyl, ethyl, n-propyl, isopropyl, n-butyl, isobutyl, sec-butyl, tert-butyl, phenyl, o-tolyl, benzyl, cyclohexyl, and —(CHR⁶)ₙ—OPO(OH)₂; R² is chosen from hydrogen, methyl, ethyl, n-propyl, isopropyl, cyclohexyl, and phenyl; and R³ is chosen from hydrogen and —PO(OH)₂. In certain embodiments of a compound of Formula (I), Y is a bond; R¹ is chosen from methyl, ethyl, n-propyl, isopropyl, n-butyl, isobutyl, sec-butyl, tert-butyl, phenyl, o-tolyl, benzyl, cyclohexyl, and —(CHR⁶)ₙ—OPO(OH)₂, wherein n is chosen from 1, 2 and 3; R² is chosen from hydrogen, methyl, ethyl, n-propyl, isopropyl, cyclohexyl, and phenyl; and R³ is chosen from hydrogen and —PO(OH)₂.

In certain embodiments, a compound of Formula (I) is chosen from:
(methylethoxycarbonyloxy)methyl (2R)-4-{[3-(acetylamino)propyl]sulfonyloxy}-3,3-dimethyl-2-hydroxy-butanoate;
(2-phenylacetyloxy)methyl (2R)-4-{[3-(acetylamino)propyl]sulfonyloxy}-3,3-dimethyl-2-hydroxy-butanoate;
(2-methylpropanoyloxy)ethyl (2R)-4-{[3-(acetylamino)propyl]sulfonyloxy}-3,3-dimethyl-2-hydroxy-butanoate;
(1S)-(2-methylpropanoyloxy)ethyl (2R)-4-{[3-(acetylamino)propyl]sulfonyloxy}-3,3-dimethyl-2-hydroxy-butanoate;
(1R)-(2-methylpropanoyloxy)ethyl (2R)-4-{[3-(acetylamino)propyl]sulfonyloxy}-3,3-dimethyl-2-hydroxy-butanoate;
ethoxycarbonyloxyethyl (2R)-4-{[3-(acetylamino)propyl]sulfonyloxy}-3,3-dimethyl-2-hydroxy-butanoate;
(1R)-1-ethoxycarbonyloxyethyl (2R)-4-{[3-(acetylamino)propyl]sulfonyloxy}-3,3-dimethyl-2-hydroxy-butanoate;
(1S)-1-ethoxycarbonyloxyethyl (2R)-4-{[3-(acetylamino)propyl]sulfonyloxy}-3,3-dimethyl-2-hydroxy-butanoate;
ethoxycarbonyloxyethyl (2R)-4-{[3-(acetylamino)propyl]sulfonyloxy}-3,3-dimethyl-2-(2-methylpropanoyloxy)-butanoate;
benzoyloxyethyl (2R)-4-{[3-(acetylamino)propyl]sulfonyloxy}-3,3-dimethyl-2-hydroxy-butanoate;
(methylethoxycarbonyloxy)ethyl (2R)-4-{[3-(acetylamino)propyl]sulfonyl oxy}-2-hydroxy-3,3-dimethylbutanoate;
(1R)-(methylethoxycarbonyloxy)ethyl (2R)-4-{[3-(acetylamino)propyl]sulfonyloxy}-2-hydroxy-3,3-dimethylbutanoate;
(1S)-(methylethoxycarbonyloxy)ethyl (2R)-4-{[3-(acetylamino)propyl]sulfonyloxy}-2-hydroxy-3,3-dimethylbutanoate;
cyclohexyloxycarbonyloxyethyl (2R)-4-{[3-(acetylamino)propyl]sulfonyloxy}-2-hydroxy-3,3-dimethylbutanoate;
cyclohexylcarbonyloxyethyl (2R)-4-{[3-(acetylamino)propyl]sulfonyloxy}-2-hydroxy-3,3-dimethylbutanoate;
2-methyl-1-(methylethoxycarbonyloxy)propyl (2R)-4-{[3-(acetylamino)propyl]sulfonyloxy}-3,3-dimethyl-2-hydroxy-butanoate;
(2-methylpropanoyloxy)ethyl (2R)-4-{[3-(acetylamino)propyl]sulfonyloxy}-3,3-dimethyl-2-[(bishydroxyphosphinyl)oxy]butanoate;
cyclohexylcarbonyloxyethyl (2R)-4-{[3-(acetylamino)propyl]sulfonyloxy}-3,3-dimethyl-2-[(bishydroxyphosphinyl)oxy]butanoate;
ethoxycarbonyloxyethyl (2R)-4-{[3-(acetylamino)propyl]sulfonyloxy}-3,3-dimethyl-2-[(bishydroxyphosphinyl)oxy]butanoate;
(methylethoxycarbonyloxy)ethyl (2R)-4-{[3-(acetylamino)propyl]sulfonyloxy}-3,3-dimethyl-2-[(bishydroxyphosphinyl)oxy]butanoate; and
   a pharmaceutically acceptable salt of any of the foregoing.

In certain embodiments, a compound of Formula (I) is chosen from:
(2-methylpropanoyloxy)ethyl (2R)-4-{[3-(acetylamino)propyl]sulfonyloxy}-3,3-dimethyl-2-hydroxy-butanoate;
(1S)-(2-methylpropanoyloxy)ethyl (2R)-4-{[3-(acetylamino)propyl]sulfonyloxy}-3,3-dimethyl-2-hydroxy-butanoate;
(1R)-(2-methylpropanoyloxy)ethyl (2R)-4-{[3-(acetylamino)propyl]sulfonyloxy}-3,3-dimethyl-2-hydroxy-butanoate;
(methylethoxycarbonyloxy)ethyl (2R)-4-{[3-(acetylamino)propyl]sulfonyloxy}-2-hydroxy-3,3-dimethylbutanoate;
(1R)-(methylethoxycarbonyloxy)ethyl (2R)-4-{[3-(acetylamino)propyl]sulfonyloxy}-2-hydroxy-3,3-dimethylbutanoate;
(1S)-(methylethoxycarbonyloxy)ethyl (2R)-4-{[3-(acetylamino)propyl]sulfonyloxy}-2-hydroxy-3,3-dimethylbutanoate;
(2-methylpropanoyloxy)ethyl (2R)-4-{[3-(acetylamino)propyl]sulfonyloxy}-3,3-dimethyl-2-[(bishydroxyphosphinyl)oxy]butanoate;
(methylethoxycarbonyloxy)ethyl (2R)-4-{[3-(acetylamino)propyl]sulfonyloxy}-3,3-dimethyl-2-[(bishydroxyphosphinyl)oxy]butanoate; and

a pharmaceutically acceptable salt of any of the foregoing.

Pharmaceutical Compositions

In other embodiments, methods of treating disorders, including but not limited to Fragile X syndrome, fragile X-associated tremor/ataxia syndrome, Down's syndrome and other forms of mental retardation, and autism is provided, comprising administering to a subject (e.g., a human) a pharmaceutical composition comprising an acamprosate prodrug of Formula (I).

Pharmaceutical compositions comprising a compound of Formula (I) may be manufactured by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers, diluents, excipients, or auxiliaries, which facilitate processing of compounds of Formula (I) or crystalline form thereof and one or more pharmaceutically acceptable vehicles into formulations that can be used pharmaceutically. Proper formulation is dependent upon the route of administration chosen. In certain embodiments, a pharmaceutical composition comprising a compound of Formula (I) or crystalline form thereof may be formulated for oral administration, and in certain embodiments for sustained release oral administration. Pharmaceutical compositions provided by the present disclosure may take the form of solutions, suspensions, emulsion, tablets, pills, pellets, capsules, capsules containing liquids, powders, sustained-release formulations, suppositories, emulsions, aerosols, sprays, suspensions, or any other form suitable for administration to a subject.

Pharmaceutical compositions provided by the present disclosure may be formulated in a unit dosage form. A unit dosage form refers to a physically discrete unit suitable as a unitary dose for subjects undergoing treatment, with each unit containing a predetermined quantity of at least one compound of Formula (I) calculated to produce an intended therapeutic effect. A unit dosage form may be for a single daily dose, for administration 2 times per day, or one of multiple daily doses, e.g., 3 or more times per day. When multiple daily doses are used, a unit dosage may be the same or different for each dose. One or more dosage forms may comprise a dose, which may be administered to a subject at a single point in time or during a time interval.

In certain embodiments, a compound of Formula (I) may be incorporated into pharmaceutical compositions to be administered orally. Oral administration of such pharmaceutical compositions may result in uptake of a compound of Formula (I) throughout the intestine and entry into the systemic circulation. Such oral compositions may be prepared in a manner known in the pharmaceutical art and comprise at least one compound of Formula (I) and at least one pharmaceutically acceptable vehicle. Oral pharmaceutical compositions may include a therapeutically effective amount of at least one compound of Formula (I) and a suitable amount of a pharmaceutically acceptable vehicle, so as to provide an appropriate form for administration to a subject.

Controlled drug delivery systems may be designed to deliver a drug in such a way that the drug level is maintained within a therapeutically effective window and effective and safe blood levels are maintained for a period as long as the system continues to deliver the drug at a particular rate. Controlled drug delivery may produce substantially constant blood levels of a drug over a period of time as compared to fluctuations observed with immediate release dosage forms. For some drugs, maintaining a constant blood and tissue concentration throughout the course of therapy is the most desirable mode of treatment. Immediate release of drugs may cause blood levels to peak above the level required to elicit a desired response, which may waste the drug and may cause or exacerbate toxic side effects. Controlled drug delivery can result in optimum therapy, and not only can reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

In certain embodiments, an oral dosage form provided by the present disclosure may be a controlled release dosage form. Controlled delivery technologies can improve the absorption of a drug in a particular region or regions of the gastrointestinal tract.

In certain embodiments, pharmaceutical compositions provided by the present disclosure may be practiced with dosage forms adapted to provide sustained release of a compound of Formula (I) upon oral administration. Sustained release oral dosage forms may be used to release drugs over a prolonged time period and are useful when it is desired that a drug or drug form be delivered to the lower gastrointestinal tract. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

Sustained release oral dosage forms may be in any appropriate form for oral administration, such as, for example, in the form of tablets, pills, or granules. Granules can be filled into capsules, compressed into tablets, or included in a liquid suspension. Sustained release oral dosage forms may additionally include an exterior coating to provide, for example, acid protection, ease of swallowing, flavor, identification, and the like.

In certain embodiments, sustained release oral dosage forms may comprise a therapeutically effective amount of a compound of Formula (I) and at least one pharmaceutically acceptable vehicle. In certain embodiments, a sustained release oral dosage form may comprise less than a therapeutically effective amount of a compound of Formula (I) and a pharmaceutically effective vehicle. Multiple sustained release oral dosage forms, each dosage form comprising less than a therapeutically effective amount of a compound of Formula (I) may be administered at a single time or over a period of time to provide a therapeutically effective dose or regimen for treating a disease in a subject. In certain embodiments, a sustained release oral dosage form comprises more than one compound of Formula (I).

Sustained release oral dosage forms provided by the present disclosure can release a compound of Formula (I) from the dosage form to facilitate the ability of a compound of Formula (I) to be absorbed from an appropriate region of the gastrointestinal tract, for example, in the small intestine or in the colon. In certain embodiments, sustained release oral dosage forms may release a compound of Formula (I) from the dosage form over a period of at least about 4 hours, at least about 8 hours, at least about 12 hours, at least about 16 hours, at least about 20 hours, and in certain embodiments, at least about 24 hours. In certain embodiments, sustained release oral dosage forms may release a compound of Formula (I) from the dosage form in a delivery pattern corresponding to about 0 wt % to about 20 wt % in about 0 to about 4 hours; about 20 wt % to about 50 wt % in about 0 to about 8 hours; about 55 wt % to about 85 wt % in about 0 to about 14 hours; and about 80 wt % to about 100 wt % in about 0 to about 24 hours; where wt % refers to the percent of the total weight of the compound in the dosage form. In certain embodiments, sustained release oral dosage forms may release a compound of Formula (I) from the dosage form in a delivery pattern corresponding to about 0 wt % to about 20 wt % in about 0 to about 4 hours; about 20 wt % to about 50 wt % in about 0 to about 8 hours; about 55 wt % to about 85 wt % in about 0 to about 14 hours; and about 80 wt % to about 100 wt % in about 0 to about 20 hours. In certain embodiments, sustained release oral dosage forms may release a compound of Formula (I) from the dosage form in a delivery pattern corresponding to about 0 wt % to about 20 wt % in about 0 to about 2 hours; about 20 wt % to about 50 wt % in about 0 to about 4 hours; about 55 wt % to about 85 wt % in about 0 to about 7 hours; and about 80 wt % to about 100 wt % in about 0 to about 8 hours.

Regardless of the specific type of controlled release oral dosage form used, a compound of Formula (I) may be released from an orally administered dosage form over a sufficient period of time to provide prolonged therapeutic concentrations of a compound of Formula (I) in the plasma and/or blood of a subject. Following oral administration, a dosage form comprising a compound of Formula (I) may provide a therapeutically effective concentration of the corresponding drug in the plasma and/or blood of a subject for a continuous time period of at least about 4 hours, of at least about 8 hours, for at least about 12 hours, for at least about 16 hours, and in certain embodiments, for at least about 20 hours following oral administration of the dosage form to the subject. The continuous time periods during which a therapeutically effective concentration of the drug is maintained may be the same or different. The continuous period of time during which a therapeutically effective plasma concentration of the drug is maintained may begin shortly after oral administration or following a time interval.

An appropriate dosage of a compound of Formula (I) or pharmaceutical composition comprising a compound of Formula (I) may be determined according to any one of several well-established protocols. For example, animal studies such as studies using mice, rats, dogs, and/or monkeys may be used to determine an appropriate dose of a pharmaceutical compound. Results from animal studies may be extrapolated to determine doses for use in other species, such as for example, humans.

Uses

In some embodiments, this disclosure is directed to the use of an acamprosate prodrug of Formula (I) in the manufacture of a medicament for use in methods of treating a mental condition such as Down's syndrome, Fragile X syndrome, and other forms of mental retardation, and/or autism. In various embodiments, the present disclosure contemplates modes of treatment and prophylaxis which utilize one or more of the compounds of Formula (I).

In certain embodiments, a compound of Formula (I) is provided for use in methods of treatment of the human or animal body by therapy; methods of treating a host suffering from Fragile X syndrome, Down's syndrome and other forms of mental retardation, or autism, which methods comprise administering to the host a therapeutically effective amount of a compound of Formula (I); pharmaceutical compositions comprising a compound of Formula (I), and a pharmaceutically acceptable carrier or diluent; or products containing a compound of Formula (I) and another therapeutic substance as a combined preparation.

Also provided herein is methods of treating a subject with Fragile X syndrome, autism, Down's syndrome, a neurological disorder and/or mental retardation in order to diminish, halt, ameliorate or prevent one or more of the neurological deficiencies or symptoms associated with the disorder (e.g., benign childhood epilepsy, temporal lobe epilepsy, visual spatial defects, anxiety, aggression, hyperactivity, agitation, repetitive behaviors, abnormal or limited social interactions, language and learning difficulties, and/or combinations thereof). In certain embodiments, children with mental retardation, autism, Down's syndrome and Fragile X syndrome can be treated with a compound of Formula (I). The children can be treated during infancy (between about 0 to about 1 year of life), childhood (the period of life between infancy and puberty) and during puberty (between about 8 years of life to about 18 years of life).

In certain embodiments, the methods disclosed herein can be used to treat adults (greater than about 18 years of life) having mental retardation, Fragile X syndrome, autism and/or Down's syndrome. In certain embodiments, anxiety and epilepsy in subjects (both children and adults) having Fragile X syndrome, autism, mental retardation and/or Down's syndrome can be treated by administering to the subjects a compound of Formula (I).

In some embodiments, a compound of Formula (I) for use in methods of treatment of a subject with Fragile X syndrome, autism, Down's syndrome, a neurological disorder or mental retardation is chosen from:
(methylethoxycarbonyloxy)methyl (2R)-4-{[3-(acetylamino)propyl]sulfonyloxy}-3,3-dimethyl-2-hydroxy-butanoate;
(2-phenylacetyloxy)methyl (2R)-4-{[3-(acetylamino)propyl]sulfonyloxy}-3,3-dimethyl-2-hydroxy-butanoate;
(2-methylpropanoyloxy)ethyl (2R)-4-{[3-(acetylamino)propyl]sulfonyloxy}-3,3-dimethyl-2-hydroxy-butanoate;
(1S)-(2-methylpropanoyloxy)ethyl (2R)-4-{[3-(acetylamino)propyl]sulfonyloxy}-3,3-dimethyl-2-hydroxy-butanoate;
(1R)-(2-methylpropanoyloxy)ethyl (2R)-4-{[3-(acetylamino)propyl]sulfonyloxy}-3,3-dimethyl-2-hydroxy-butanoate;
ethoxycarbonyloxyethyl (2R)-4-{[3-(acetylamino)propyl]sulfonyloxy}-3,3-dimethyl-2-hydroxy-butanoate;
(1R)-1-ethoxycarbonyloxyethyl (2R)-4-{[3-(acetylamino)propyl]sulfonyloxy}-3,3-dimethyl-2-hydroxy-butanoate;
(1S)-1-ethoxycarbonyloxyethyl (2R)-4-{[3-(acetylamino)propyl]sulfonyloxy}-3,3-dimethyl-2-hydroxy-butanoate;
ethoxycarbonyloxyethyl (2R)-4-{[3-(acetylamino)propyl]sulfonyloxy}-3,3-dimethyl-2-(2-methylpropanoyloxy)-butanoate;
benzoyloxyethyl (2R)-4-{[3-(acetylamino)propyl]sulfonyloxy}-3,3-dimethyl-2-hydroxy-butanoate;
(methylethoxycarbonyloxy)ethyl (2R)-4-{[3-(acetylamino)propyl]sulfonyloxy}-2-hydroxy-3,3-dimethylbutanoate;
(1R)-(methylethoxycarbonyloxy)ethyl (2R)-4-{[3-(acetylamino)propyl]sulfonyloxy}-2-hydroxy-3,3-dimethylbutanoate;
(1S)-(methylethoxycarbonyloxy)ethyl (2R)-4-{[3-(acetylamino)propyl]sulfonyloxy}-2-hydroxy-3,3-dimethylbutanoate;
cyclohexyloxycarbonyloxyethyl (2R)-4-{[3-(acetyl amino)propyl]sulfonyloxy}-2-hydroxy-3,3-dimethylbutanoate;
cyclohexylcarbonyloxyethyl (2R)-4-{[3-(acetylamino)propyl]sulfonyloxy}-2-hydroxy-3,3-dimethylbutanoate;
2-methyl-1-(methylethoxycarbonyloxy)propyl (2R)-4-{[3-(acetylamino)propyl]sulfonyloxy}-3,3-dimethyl-2-hydroxy-butanoate;
(2-methylpropanoyloxy)ethyl (2R)-4-{[3-(acetylamino)propyl]sulfonyloxy}-3,3-dimethyl-2-[(bishydroxyphosphinyl)oxy]butanoate;
cyclohexylcarbonyloxyethyl (2R)-4-{[3-(acetylamino)propyl]sulfonyloxy}-3,3-dimethyl-2-[(bishydroxyphosphinyl)oxy]butanoate;
ethoxycarbonyloxyethyl (2R)-4-{[3-(acetylamino)propyl]sulfonyloxy}-3,3-dimethyl-2-[(bishydroxyphosphinyl)oxy]butanoate;
(methylethoxycarbonyloxy)ethyl (2R)-4-{[3-(acetylamino)propyl]sulfonyloxy}-3,3-dimethyl-2-[(bishydroxyphosphinyl)oxy]butanoate; and

a pharmaceutically acceptable salt of any of the foregoing.

In other embodiments, a compound of Formula (I) for use in methods of treatment of a subject with Fragile X syndrome, autism, Down's syndrome, a neurological disorder or mental retardation is chosen from:
(2-methylpropanoyloxy)ethyl (2R)-4-{[3-(acetylamino)propyl]sulfonyloxy}-3,3-dimethyl-2-hydroxy-butanoate;
(1S)-(2-methylpropanoyloxy)ethyl (2R)-4-[3-(acetylamino)propyl]sulfonyloxy-3,3-dimethyl-2-hydroxy-butanoate;
(1R)-(2-methylpropanoyloxy)ethyl (2R)-4-{[3-(acetylamino)propyl]sulfonyloxy}-3,3-dimethyl-2-hydroxy-butanoate;
(methylethoxycarbonyloxy)ethyl (2R)-4-{[3-(acetylamino)propyl]sulfonyloxy}-2-hydroxy-3,3-dimethylbutanoate;
(1R)-(methylethoxycarbonyloxy)ethyl (2R)-4-{[3-(acetylamino)propyl]sulfonyloxy}-2-hydroxy-3,3-dimethylbutanoate;
(1S)-(methylethoxycarbonyloxy)ethyl (2R)-4-{[3-(acetylamino)propyl]sulfonyloxy}-2-hydroxy-3,3-dimethylbutanoate;
(2-methylpropanoyloxy)ethyl (2R)-4-{[3-(acetylamino)propyl]sulfonyloxy}-3,3-dimethyl-2-[(bishydroxyphosphinyl)oxy]butanoate;
(methylethoxycarbonyloxy)ethyl (2R)-4-{[3-(acetylamino)propyl]sulfonyloxy}-3,3-dimethyl-2-[(bishydroxyphosphinypoxy]butanoate; and
   a pharmaceutically acceptable salt of any of the foregoing.

The amount of a compound of Formula (I) that will be effective in the treatment of a disease in a subject will depend, in part, on the nature of the condition and can be determined by standard clinical techniques known in the art. In addition, in vitro or in vivo assays may be employed to help identify optimal dosage ranges. A therapeutically effective amount of compound of Formula (I) to be administered may also depend on, among other factors, the subject being treated, the weight of the subject, the severity of the disease, the manner of administration, and the judgment of the prescribing physician.

In certain embodiments, the methods of treatment comprise administering to the subject an effective amount of one or more compounds of Formula (I), alone or in combinations thereof. In other embodiments, a compound of Formula (I) is administered in a dose ranging from about 1 to about 200 mg/kg body weight/day. In some embodiments, a compound of Formula (I) is administered in a dose ranging from about 5 to about 100 mg/kg body weight/day. In other embodiments, a compound of Formula (I) is administered in a dose ranging from about 5 to about 50 mg/kg body weight/day.

For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in subjects. One having ordinary skill in the art may optimize administration to humans based on animal data.

A dose may be administered in a single dosage form or in multiple dosage forms. When multiple dosage forms are used, the amount of compound contained within each dosage form may be the same or different. The amount of a compound of Formula (I) contained in a dose may depend on the route of administration and whether the disease in a subject is effectively treated by acute, chronic, or a combination of acute and chronic administration. In some embodiments, compounds of Formula (I) are dosed by oral administration.

In certain embodiments an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, compounds of Formula (I) may exhibit a high therapeutic index. The data obtained from, for example, cell culture assays and/or animal studies, may be used in formulating a dosage range that is not toxic for use in humans. A dose of a compound of Formula (I) provided by the present disclosure may be within a range of circulating concentrations in for example the blood, plasma, or central nervous system, that include the effective dose and that exhibits little or no toxicity.

In certain embodiments, compounds of Formula (I) can be used in combination therapy with at least one other therapeutic agent to treat Fragile X syndrome, Down's syndrome and other forms of mental retardation, or autism. Compounds of Formula (I) and the at least one other therapeutic agent(s) may act additively or, in certain embodiments, synergistically. In certain embodiments, compounds of Formula (I) can be administered concurrently with the administration of another therapeutic agent. In certain embodiments, compounds of Formula (I) may be administered prior or subsequent to administration of another therapeutic agent. The at least one other therapeutic agent may be effective for treating the same or different disease or disorder.

In some embodiments, compounds of Formula (I) can be used in combination therapy with mGluR antagonists to treat Fragile X syndrome, Down's syndrome and other forms of mental retardation, or autism. Suitable mGluR antagonists are Group I mGluR antagonists including, for example, 2-methyl-6-(phenylethynyl)-pyridine (MPEP), (E)-6-methyl-2-styryl-pyridine (SIB 1893), 6-methyl-2-(phenylazo)-3-pyridinol and α-methyl-4-carboxyphenylglycine (MCPG). Other Group I mGluR antagonists for use are described in U.S. Pat. Nos. 6,890,931 and 6,916,821. Yet other suitable mGluR antagonists are mGluR5 antagonists described in WO 01/66113, WO 01/32632, WO 01/14390, WO 01/08705, WO 01/05963, WO 01/02367, WO 01/02342, WO 01/02340, WO 00/20001, WO 00/73283, WO 00/69816, WO 00/63166, WO 00/26199, WO 00/26198, EP-A-0807621, WO 99/54280, WO 99/44639, WO 99/26927, WO 99/08678, WO 99/02497, WO 98/45270, WO 98/34907, WO 97/48399, WO 97/48400, WO 97/48409, WO 98/53812, WO 96/15100, WO 95/25110, WO 98/06724, WO 96/15099 WO 97/05109, WO 97/05137, and U.S. Pat. Nos. 6,218,385, 5,672,592, 5,795,877, 5,863,536, 5,880,112 and 5,902,817.

In other embodiments, compounds of Formula (I) can be used in combination therapy with antipsychotic agents to treat Fragile X syndrome, Down's syndrome and other forms of mental retardation, or autism. Antipsychotic agents, including atypical antipsychotic compounds for use in combination treatment can include, for example, abaperidone, acetophenazine maleate, alentemol hydrobromide, alpertine, amisulpride, aripiprazole, azaperone, batelapine maleate, benperidol, benzindopyrine hydrochloride, brofoxine, bromperidol, butaclamol hydrochloride, butaperazine, carphenazine maleate, carvotroline hydrochloride, chlorpromazine, chlorprothixene, cinperene, cintriamide, clomacran phosphate, clopenthixol, clopimozide, clopipazan mesylate, cloroperone hydrochloride, clothiapine, clothixamide maleate, clozapine, cyclophenazine hydrochloride, droperidol, etazolate hydrochloride, fenimide, flucindole, flumezapine, fluphenazine decanoate, fluphenazine enanthate, fluphenazine hydrochloride, fluspiperone, fluspirilene, flutroline, gevotroline hydrochloride, halopemide, haloperidol, iloperidone, imidoline hydrochloride, lenperone, loxapine, mazapertine succinate, mesoridazine, metiapine, milenperone, milipertine, molindone hydrochloride, naranol hydrochloride, neflumozide hydrochloride, nemonapride, ocaperidone, olanzapine, oxiperomide, penfluridol, pentiapine maleate, perospirone, perphenazine, pimozide, pinoxepin hydrochloride, pipamperone, piperacetazine, pipotiazine palmnitate, piquindone hydrochloride, prochlorperazine edisylate, prochlorperazine maleate, promazine hydrochloride, quetiapine, remoxipride, remoxipride hydrochloride, risperidone, rimcazole hydrochloride, seperidol hydrochloride, sertindole, setoperone, spiperone, sulpiride, thioridazine, thiothixene, thorazine, tioperidone hydrochloride, tiospirone hydrochloride, trifluoperazine hydrochloride, trifluperidol, triflupromazine, ziprasidone hydrochloride, zotepine, zuclopenthixol, as well as analogs, derivatives and combinations thereof.

In other embodiments, compounds of Formula (I) can be used in combination therapy with at least one compound selected from the group consisting of a GABA_{B} receptor agonist, a muscarinic receptor antagonist, a stimulant, a nicotinic receptor agonist, an endocannabinoid receptor antagonist, an AMPA agonist, an antidepressant, an α2-adrenergic agonist, or an anticonvulsant to treat Fragile X syndrome, Down's syndrome and other forms of mental retardation, or autism. In some embodiments, the GABA_{B} receptor agonist is baclofen, R-baclofen, or a prodrug thereof, for example as disclosed in U.S. Pat. Nos. 7,109,239 and 7,300,956. In other embodiments, the muscarinic receptor antagonist is atropine, benztropine, biperiden, dicyclomine, ipratroprium, procyclidine, scopolamine, tiotropium, telenzepine or trihexyphenidyl. In other embodiments, the stimulant is amantadine, bupropion, atomoxetine, modafinil, caffeine, methylphenidate, nicotine, pseudoephedrine, and amphetamine, as well as metabolites, isomers or prodrugs thereof.

### EXAMPLES

The following examples describe methods of synthesis of compounds of Formula (I), properties of compounds of Formula (I), and uses of compounds of Formula (I). It will be apparent to those skilled in the art that many modifications, both to materials and methods, may be practiced without departing from the scope of the disclosure.

### Example 1

### General Experimental Protocols

All reagents and solvents were purchased from commercial suppliers and used without further purification or manipulation.

Proton NMR spectra (400 MHz) were recorded on a Varian AS 400 NMR spectrometer equipped with an autosampler and data processing computation. CDCl₃ (99.8% D), DMSO-d⁶ (99.9% D), or MeOH-d⁴ (99.8+% D) were used as solvents unless otherwise noted. The CHCl₃, DMSO-d⁵, or MeOH-d³ solvent signals were used for calibration of the individual spectra. Determination of enantiomeric excess (e.e.) of intermediates was accomplished by ¹H NMR spectroscopy in the presence of the diamagnetic enantiomerically pure chiral co-solvent (R)-(−)-2,2,2-trifluoro-1-(9-anthryl)ethanol (Pirkle-alcohol) and in comparison to ¹H NMR spectra of the corresponding racemic samples under similar conditions. Analytical thin layer chromatography (TLC) was performed using Whatman, Schleicher & Schuell TLC. MK6F silica gel plates (2.5×7.5 cm, 250 μm layer thickness). Dyeing or staining reagents for TLC detection and visualization were prepared according methods known in the art. Ozonolysis reactions were performed using a Weisbach Standard T-series ozone generator. Analytical LC/MS was performed on a Waters 2790 separation module equipped with a Waters Micromass QZ mass spectrometer, a Waters 996 photodiode detector, and a Merck Chromolith UM2072-027 or Phenomenex Luna C-18 analytical column. Mass-guided preparative HPLC purification of final compounds was performed on an instrument equipped with a Waters 600 controller, ZMD Micromass spectrometer, a Waters 2996 photodiode array detector, and a Waters 2700 Sample Manager. Acetonitrile/water gradients containing 0.05% formic acid were used as eluent in both analytical and preparative HPLC procedures. Compound isolation from aqueous solvent mixtures, e.g., acetonitrile/water/0.05% formic acid, was accomplished by primary lyophilization (freeze drying) of the frozen solutions under reduced pressure at room temperature using manifold freeze dryers such as Heto Drywinner DW 6-85-1, Heto FD4, or VIRTIS Freezemobile 25 ES equipped with a high vacuum pump. Optionally, and if the isolated compound had ionizable functional groups such as an amino group or a carboxylic acid, the lyophilization process was conducted in the presence of a slight excess of one molar (1.0 M) hydrochloric acid to yield the purified compound(s) as the corresponding hydrochloride salt (HCl-salt) or the corresponding protonated free carboxylic acid. Additionally, the separation of diastereomers was performed with a UV-guided preparative HPLC using a 250×21.2 mm chiral column Chiralpak IA (Daicel Chemical Industries, Ltd.) and a mixture of ethanol (EtOH) and hexanes (H×n) (EtOH/H×n=1:9) containing up to 0.1% formic acid or trifluoroacetic acid (TFA). The flow rate of the mobile phase was 20 mL/min at room temperature. The mixture of diastereomers was dissolved in a mixture of hexane (Hex) and isopropanol (iPrOH) (1:1). The injection volume was 20-60 mg/1,000 μL. Compounds were obtained following concentration under reduced pressure using a rotary evaporator.

### Example 2

### Synthesis of Compounds of Formula (I)

Compounds of Formula (I) were synthesized according to methods disclosed in U.S. Patent Application No. 2009/0076147 and characterized by ¹H NMR spectroscopy and mass spectrometry. Certain embodiments of compounds of Formula (I) and their corresponding analytical data are reported below.

### (Methylethoxycarbonyloxy)methyl (2R)-4-{[3-(acetylamino)propyl]sulfonyloxy}-3,3-dimethyl-2-hydroxy-butanoate

¹H NMR (400 MHz, CDCl₃): δ=1.01 (s, 3H), 1.13 (s, 3H), 1.33-1.35 (m, 6H), 2.00 (s, 3H), 2.07-2.11 (m, 2H), 3.13-3.26 (m, 2H), 3.35-3.45 (m, 2H), 3.97 (d, J=9.2 Hz, 1H), 4.12 (s, 1H), 4.16 (d, J=9.6 Hz, 1H), 4.94 (sept., J=6.4 Hz, 1H), 5.79 (d, J=5.6 Hz, 1H), 5.81-5.89 (br. m, 1H), 5.91 (d, J=5.6 Hz, 1H), ppm. MS (ESI) m/z: 428.04 (M+H)⁺, 450.02 (M+Na)⁺.

### (2-Phenylacetyloxy)methyl (2R)-4-{[3-(acetylamino)propyl]sulfonyloxy}-3,3-dimethyl-2-hydroxy-butanoate

¹H NMR (400 MHz, CDCl₃): δ=0.91 (s, 3H), 1.00 (s, 3H), 1.98 (s, 3H), 2.03-2.10 (m, 2H), 3.10-3.23 (m, 2H), 3.26 (br., 1H), 3.32-3.45 (m, 2H), 3.70 (s, 2H), 3.90 (d, J=9.6 Hz, 1H), 4.07 (s, 1H), 4.10 (d, J=9.2 Hz, 1H), 5.79 (d, J=5.6 Hz, 1H), 5.85 (br. m, 1H), 5.93 (d, J=5.6 Hz, 1H) 7.26-7.35 (m, 5H) ppm. MS (ESI) m/z: 460.05 (M+H)⁺, 481.99 (M+Na)⁺.

### (2-Methylpropanoyloxy)ethyl (2R)-4-{[3-(acetylamino)propyl]sulfonyloxy}-3,3-dimethyl-2-hydroxy-butanoate

¹H NMR (400 MHz, CDCl₃, both diastereomers): δ=0.95-0.99 (2s, 3H), 1.12-1.14 (2s, 3H), 1.17-1.20 (m, 6H), 1.55-1.57 (2d, J=5.6, 5.6 Hz, 3H), 1.99-2.00 (2s, 3H), 2.04-2.12 (m, 2H), 2.53-2.62 (m, 1H), 3.13-3.23 (m, 2H), 3.26-3.29 (br. m, 1H), 3.34-3.45 (m, 2H), 3.91-3.97 (2d, J=9.6, 9.2 Hz, 1H), 4.03-4.06 (m, 1H), 4.14-4.19 (2d, J=9.2, 9.2 Hz, 1H), 5.96-6.01 (br. m, 1H), 6.91-6.97 (m, 1H) ppm. MS (ESI) m/z: 426.04 (M+H)⁺, 447.99 (M+Na)⁺.

### (1S)-(2-Methylpropanoyloxy)ethyl (2R)-4-{[3-(acetylamino)propyl]sulfonyloxy}-3,3-dimethyl-2-hydroxy-butanoate

¹H NMR (400 MHz, CDCl₃): δ=1.00 (s, 3H), 1.14 (s, 3H), 1.19 (d, J=7.2 Hz, 6H), 1.56 (d, J=5.6 Hz, 3H), 2.02 (s, 3H), 2.05-2.12 (m, 2H), 2.58 (sept., J=6.8 Hz, 1H), 3.12-3.25 (m, 2H), 3.33-3.48 (m, 2H), 3.96 (d, J=8.8 Hz, 1H), 4.05 (s, 1H), 4.15 (d, J=9.2 Hz, 1H), 5.98-6.03 (br. m, 1H), 6.94 (q, J=5.6 Hz, 1H) ppm. MS (ESI) m/z: 426.03 (M+H)⁺, 447.99 (M+Na)⁺.

### (1R)-(2-Methylpropanoyloxy)ethyl (2R)-4-{[3-(acetylamino)propyl]sulfonyloxy}-3,3-dimethyl-2-hydroxy-butanoate

¹H NMR (400 MHz, CDCl₃): δ=0.92 (s, 3H), 1.08 (s, 3H), 1.15 (d, J=7.2 Hz, 6H), 1.52 (d, J=5.6 Hz, 3H), 1.96 (s, 3H), 2.01-2.08 (m, 2H), 2.54 (sept., J=6.8 Hz, 1H), 3.12-3.22 (m, 2H), 3.28-3.42 (m, 2H), 3.88 (d, J=9.2 Hz, 1H), 4.03 (s, 1H), 4.16 (d, J=9.2 Hz, 1H), 6.37-6.39 (br. m, 1H), 6.91 (q, J=5.6 Hz, 1H) ppm. MS (ESI) m/z: 426.03 (M+H)⁺, 447.99 (M+Na)⁺.

Ethoxycarbonyloxyethyl (2R)-4-{[3-(acetylamino)propyl]sulfonyloxy}-3,3-dimethyl-2-hydroxy-butanoate

¹H NMR (400 MHz, CDCl₃, both diastereomers): δ=0.99-1.01 (2s, 3H), 1.13-1.15 (2s, 3H), 1.32-1.37 (m, 3H), 1.59-1.62 (m, 3H), 2.00-2.01 (2s, 3H), 2.04-2.12 (m, 2H), 3.12-3.26 (m, 3H), 3.33-3.47 (m, 2H), 3.94-3.98 (m, 1H), 4.06-4.08 (2d, J=6.8, 6.0 Hz, 1H), 4.14-4.19 (2d, J=8.8, 8.8 Hz, 1H), 4.23-4.28 (m, 2H), 5.85-5.88 (br. m, 1H), 6.85 (m, 1H) ppm. MS (ESI) m/z: 428.14 (M+H)⁺, 450.14 (M+Na)⁺.

### (1R)-1-Ethoxycarbonyloxyethyl (2R)-4-{[3-(acetylamino)propyl]sulfonyloxy}-3,3-dimethyl-2-hydroxy-butanoate

Earlier eluting diastereomer (arbitrary stereochemical assignment) D.e.>98% (by ¹H NMR spectroscopy, 400 MHz, CDCl₃ and analytical LC/UV). ¹H NMR (400 MHz, CDCl₃): δ=0.99 (s, 3H), 1.13 (s, 3H), 1.34 (t, J=7.6 Hz, 3H), 1.59 (d, J=5.2 Hz, 3H), 2.01 (s, 3H), 2.06-2.13 (m, 2H), 3.14-3.27 (m, 2H), 3.35-3.48 (m, 2H), 3.95 (d, J=9.6 Hz, 1H), 4.07 (s, 1H), 4.19 (d, J=9.2 Hz, 1H), 4.25 (q, J=7.2 Hz, 2H), 5.86-5.98 (br. m, 1H), 6.85 (q, J=5.6 Hz, 1H) ppm. MS (ESI) m/z: 428.14 (M+H)⁺, 450.14 (M+Na)⁺.

### (1S)-1-Ethoxycarbonyloxyethyl (2R)-4-{[3-(acetylamino)propyl]sulfonyloxy}-3,3-dimethyl-2-hydroxy-butanoate

Later eluting diastereomer (arbitrary stereochemical assignment) D.e.>98% (by ¹H NMR spectroscopy, 400 MHz, CDCl₃ and analytical LC/UV). ¹H NMR (400 MHz, CDCl₃): δ=1.01 (s, 3H), 1.14 (s, 3H), 1.35 (t, J=7.6 Hz, 3H), 1.60 (d, J=5.6 Hz, 3H), 2.01 (s, 3H), 2.05-2.12 (m, 2H), 3.12-3.25 (m, 2H), 3.33-3.46 (m, 2H), 3.97 (d, J=9.2 Hz, 1H), 4.07 (s, 1H), 4.15 (d, J=9.2 Hz, 1H), 4.25 (q, J=7.2 Hz, 2H), 5.92-5.98 (br. m, 1H), 6.84 (q, J=5.6 Hz, 1H) ppm. MS (ESI) m/z: 428.13 (M+H)⁺, 450.14 (M+Na)⁺.

### Ethoxycarbonyloxyethyl (2R)-4-{[3-(acetylamino)propyl]sulfonyloxy}-3,3-dimethyl-2-(2-methylpropanoyloxy)-butanoate

¹H NMR (400 MHz, CDCl₃, both diastereomers): δ=1.11 (m, 3H), 1.12 (m, 3H), 1.19-1.23 (m, 6H), 1.327 (t, J=7.2 Hz, 3H), 1.49-1.57 (2d, J=5.6, 5.6 Hz, 3H), 1.99 (s, 3H), 2.03-2.10 (m, 2H), 2.68-2.70 (m, 1H), 3.16-3.22 (m, 2H), 3.37-3.43 (m, 2H), 3.96-3.99 (2d, J=9.6, 9.6 Hz, 1H), 4.11-4.14 (2d, J=10.0, 10.0 Hz, 1H), 4.20-4.24 (m, 2H), 4.74-4.77 (2s, 1H), 6.02-6.08 (br. m, 1H), 6.76-6.82 (m, 11-1) ppm. MS (ESI) m/z 498.17 (M+H)⁺, 520.19 (M+Na)⁺.

### Benzoyloxyethyl (2R)-4-{[3-(acetylamino)propyl]sulfonyloxy}-3,3-dimethyl-2-hydroxy-butanoate

¹H NMR (400 MHz, CDCl₃, both diastereomers): δ=0.95-1.01 (2s, 3H), 1.11-1.14 (2s, 3H), 1.68-1.70 (2d, J=5.6, 5.6 Hz, 3H), 1.97-1.99 (2s, 3H), 2.00-2.08 (m, 2H), 3.11-3.21 (m, 2H), 3.30-3.41 (m, 2H), 3.45-3.55 (2d, J=6.4, 6.8 Hz, 1H), 3.90-3.97 (2d, J=9.2, 8.8 Hz, 1H), 4.06-4.12 (m, 1H), 4.16-4.19 (m, 1H), 6.10-6.18 (br. m, 1H), 7.17-7.22 (m, 1H), 7.43-7.47 (m, 2H), 7.57-7.62 (m, 1H), 8.00-8.05 (m, 2H) ppm. MS (ESI) m/z 460.05 (M+H)⁺, 481.99 (M+Na)⁺.

### (Methylethoxycarbonyloxy)ethyl (2R)-4-{[3-(acetylamino)propyl]sulfonyloxy}-2-hydroxy-3,3-dimethylbutanoate

¹H NMR (400 MHz, DMSO-d⁶, both diastereomers): δ=0.89-0.91 (2s, 3H), 0.93-0.95 (2s, 3H), 1.21-1.24 (m, 6H), 1.45-1.47 (2d, J=5.2, 5.2 Hz, 3H), 1.78-1.83 (m, 5H), 3.10-3.15 (m, 2H), 3.30-3.33 (m, 2H), 3.89-3.93 (m, 2H), 4.05-4.08 (2d, J=9.2, 9.6 Hz, 1H), 4.73-4.82 (m, 1H), 5.78-5.85 (2d, J=6.0, 6.0 Hz, 1H), 6.65-6.71 (m, 1H), 7.89-7.92 (br. m, 1H) ppm. MS (ESI) m/z: 442.08 (M+H)⁺, 464.02 (M+Na)⁺.

### (1R)-(Methylethoxycarbonyloxy)ethyl (2R)-4-{[3-(acetylamino)propyl]sulfonyloxy}-2-hydroxy-3,3-dimethylbutanoate

Earlier eluting diastereomer (arbitrary stereochemical assignment) D.e.>98% (by ¹H NMR spectroscopy, 400 MHz, CDCl₃ and LC/UV). ¹H NMR (400 MHz, CDCl₃): δ=0.96 (s, 3H), 1.11 (s, 3H), 1.31 (t, J=6.0 Hz, 6H), 1.57 (d, J=5.2 Hz, 3H), 1.99 (s, 3H), 2.04-2.11 (m, 2H), 3.13-3.26 (m, 2H), 3.32-3.46 (m, 2H), 3.91 (d, J=9.2 Hz, 1H), 4.06 (s, 1H), 4.17 (d, J=9.2 Hz, 1H), 4.84-4.92 (m, 1H), 6.13-6.17 (br. m, 1H), 6.83 (q, J=5.2 Hz, 1H) ppm. MS (ESI) m/z: 442.08 (M+H)⁺, 464.02 (M+Na)⁺.

### (1S)-(Methylethoxycarbonyloxy)ethyl (2R)-4-{[3-(acetylamino)propyl]sulfonyloxy}-2-hydroxy-3,3-dimethylbutanoate

Later eluting diastereomer (arbitrary stereochemical assignment) D.e.>98% (by ¹H NMR spectroscopy, 400 MHz, CDCl₃ and LC/UV). ¹H NMR (400 MHz, CDCl₃): δ=1.00 (s, 3H), 1.14 (s, 3H), 1.32-1.34 (dd, J=6.4 Hz, 6.4 Hz, 6H), 1.59 (d, J=4.8 Hz, 3H), 2.01 (s, 3H), 2.04-2.11 (m, 2H), 3.13-3.25 (m, 2H), 3.33-3.46 (m, 2H), 3.95 (d, J=9.6 Hz, 1H), 4.06 (s, 1H), 4.15 (d, J=9.2 Hz, 1H), 4.90 (sept, J=6.0 Hz, 1H), 6.18-6.25 (br. m, 1H), 6.83 (q, J=5.2 Hz, 1H) ppm. MS (ESI) m/z: 442.08 (M+H)⁺, 464.02 (M+Na)⁺.

### Cyclohexyloxycarbonyloxyethyl (2R)-4-{[3-(acetylamino)propyl]sulfonyloxy}-2-hydroxy-3,3-dimethylbutanoate

¹H NMR (400 MHz, DMSO-d⁶, both diastereomers): δ=0.89-0.90 (2s, 3H), 0.93-0.94 (2s, 3H), 1.31-1.47 (br. m, 9H), 1.60-1.68 (br. m, 2H), 1.78-1.83 (br. m, 7H), 3.10-3.15 (m, 2H), 3.28-3.35 (m, 2H), 3.89-3.92 (m, 2H), 4.05-4.08 (2d, J=9.2, 9.6 Hz, 1H), 4.52-4.58 (m, 1H), 5.78-5.85 (2d, J=5.6, 5.6 Hz, 1H), 6.66-6.72 (m, 1H), 7.89-7.92 (br. m, 1H) ppm. MS (ESI) m/z: 482.07 (M+H)⁺, 503.83 (M+Na)⁺.

### Cyclohexylcarbonyloxyethyl (2R)-4-{[3-(acetylamino)propyl]sulfonyloxy}-2-hydroxy-3,3-dimethylbutanoate

¹H NMR (400 MHz, DMSO-d⁶, both diastereomers): δ=0.92-0.94 (2s, 3H), 0.97-0.98 (2s, 3H), 1.20-1.42 (br. m, 5H), 1.46-1.48 (2d, J=5.6, 5.6 Hz, 3H), 1.55-1.62 (br. m, 1H), 1.66-1.70 (br. m, 2H), 1.80-1.87 (m, 7H), 2.33-2.40 (m, 1H), 3.14-3.19 (m, 2H), 3.34-3.37 (m, 2H), 3.92-3.96 (m, 2H), 4.09-4.12 (2d, J=9.2, 9.6 Hz, 1H), 5.50-6.87 (2d, J=5.2, 5.6 Hz, 1H), 6.81-6.87 (m, 1H), 7.95-7.98 (br. m, 1H) ppm. MS (ESI) m/z: 466.10 (M+H)⁺, 488.07 (M+Na)⁺.

### 2-Methyl-1-(methylethoxycarbonyloxy)propyl (2R)-4-{[3-(acetylamino)propyl]sulfonyloxy}-3,3-dimethyl-2-hydroxy-butanoate

¹H NMR (400 MHz, CDCl₃, both diastereomers): δ=0.96-1.00 (2s, 3H), 1.01-1.05 (m, 6H), 1.14-1.16 (2s, 3H), 1.30-1.34 (m, 6H), 1.99-2.01 (m, 4H), 2.03-2.15 (m, 2H), 3.13-3.24 (m, 2H), 3.33 (d, J=6.8 Hz, 1H), 3.35-3.45 (m, 2H), 3.90-3.96 (2d, J=9.2, 9.6 Hz, 1H), 4.07-4.10 (2d, J=6.8, 6.8 Hz, 1H), 4.16-4.21 (2d, J=9.2, 9.2 Hz, 1H), 4.85-4.94 (m, 1H), 5.98-6.03 (br. m, 1H), 6.58-6.63 (dd, J=4.4 Hz, 4.8 Hz, 1H) ppm. MS (ESI) m/z 470.07 (M+H)⁺, 492.06 (M+Na)⁺.

### (2-Methylpropanoyloxy)ethyl (2R)-4-{[3-(acetylamino)propyl]sulfonyloxy}-3,3-dimethyl-2-[(bishydroxyphosphinyl)oxy]butanoate

¹H NMR (400 MHz, CDCl₃, both diastereomers): δ=1.01-1.02 (2s, 3H), 1.07 (s, 3H), 1.13-1.16 (m, 6H), 1.51-1.53 (m, 3H), 2.11-2.13 (m, 5H), 2.49-2.61 (m, 1H), 3.27-3.43 (m, 4H), 3.94-3.97 (m, 1H), 4.09-4.13 (m, 1H), 4.51 (d, J=8.4 Hz, 1H), 6.88-6.94 (m, 1H), 7.64-7.70 (br. m, 1H), 7.85-8.15 (br. m, 2H) ppm. MS (ESI) m/z 506.04 (M+H)⁺, 528.04 (M+Na)⁺.

### Cyclohexylcarbonyloxyethyl (2R)-4-{[3-(acetylamino)propyl]sulfonyloxy}-3,3-dimethyl-2-[(bishydroxyphosphinyl)oxy]butanoate

¹H NMR (400 MHz, CD₃OD, both diastereomers): δ=1.09 (s, 3H), 1.10-1.11 (s, 3H), 1.26-1.49 (br. m, 5H), 1.53-1.54 (2d, J=5.2, 5.6 Hz, 3H), 1.66-1.69 (br. m, 1H), 1.75-1.79 (br. m, 2H), 1.90-1.94 (br. m, 2H), 1.98 (s, 3H), 2.01-2.08 (m, 2H), 2.34-2.42 (m, 1H), 3.29-3.35 (m, 4H), 4.06-4.13 (m, 2H), 4.51-4.54 (2d, J=8.0, 8.0 Hz, 1H), 6.92-6.98 (m, 1H) ppm. MS (ESI) m/z 546.04 (M+H)⁺, 567.99 (M+Na)⁺.

### Ethoxycarbonyloxyethyl (2R)-4-{[3-(acetylamino)propyl]sulfonyloxy}-3,3-dimethyl-2-[(bishydroxyphosphinyl)oxy]butanoate

¹H NMR (400 MHz, CD₃OD, both diastereomers): δ=0.97-0.98 (2s, 3H), 1.01 (s, 3H), 1.20 (t, J=7.2 Hz, 3H), 1.43-1.45 (2d, J=5.2, 4.8 Hz, 3H), 1.86 (s, 3H), 1.89-1.96 (m, 2H), 3.16-3.23 (m, 4H), 3.94-4.00 (m, 2H), 4.08-4.14 (m, 2H), 4.40-4.42 (2d, J=8.4, 8.0 Hz, 1H), 6.68-6.73 (m, 1H) ppm. MS (ESI) m/z 507.76 (M+H)⁺, 529.71 (M+Na)⁺.

### (Methylethoxycarbonyloxy)ethyl (2R)-4-{[3-(acetylamino)propyl]sulfonyloxy}-3,3-dimethyl-2-[(bishydroxyphosphinypoxy]butanoate

¹H NMR (400 MHz, CD₃OD, both diastereomers): δ=1.07 (s, 3H), 1.09-1.10 (2s, 3H), 1.28-1.30 (2d, J=6.4, 6.0 Hz, 6H), 1.52-1.54 (2d, J=5.6, 5.6 Hz, 3H), 1.99 (s, 3H), 1.99-2.06 (m, 2H), 3.26-3.23 (m, 4H), 4.03-4.10 (m, 2H), 4.49-4.52 (2d, J=8.4, 8.0 Hz, 1H), 4.83-4.88 (m, 1H), 6.76-6.83 (m, 1H) ppm. MS (ESI) m/z 521.94 (M+H)⁺, 543.93 (M+Na)⁺.

### Example 3

### Treatment of Fragile X Knockout Mice with Acamprosate Prodrugs in Audiogenic Seizure Assay

The goal of the experiment is to determine if the sensitivity to audiogenic seizures are reduced in Fmr1 KO mice following administration of acamprosate prodrugs of Formula (I). The protocol is adapted from methods described in Yan et al., Neuropharmacology 2005, 49, 1053-66. Fmr1 KO mice receive vehicle or test article (at doses from 1 mg/kg to 200 mg/kg) by oral gavage (N=10 animals per dose group) 1 hour prior to testing for audiogenic seizures. Male FVB/NJ (“FVB”) mice of 14 to 25 days of age are exposed to a high intensity siren of frequency peak 1800-6300 Hz at an average sound pressure level of 125 dB at 11 cm (Personal Alarm, Model 49-417, Tandy Corporation) in an empty, transparent plastic box (28×17.5×12 cm) with a sound absorbent tile lid under which the siren is mounted. The alarm is powered from a DC converter in order to ensure that sound pressure levels are maintained above 115 dB. After 1 min the alarm sound is turned on for two minutes. After a two-minute exposure to the alarm, mice are given another minute of no sound followed by a second two-minute alarm. The presence of seizures as defined by ‘non-startling,’ wild-running or tonic/clonic seizures is recorded (mice typically do not display a seizure during the first alarm period). The primary endpoint is frequency of status epilepticus, a sustained tonic seizure most often resulting in respiratory arrest and death. In addition, the latency to wild-running and/or tonic/clonic seizures is recorded. The percentage of mice displaying seizures is calculated. Compounds of Formula (I) produce a statistically significant reduction in audiogenic seizure frequency relative to controls, with active compounds causing a greater than 50% reduction in seizures.

### Example 4

### Treatment of Fragile X Knockout Mice with Acamprosate Prodrugs in Open Field Testing

The goal of the experiment is to determine if open-field activity in Fmr1 KO mice is altered following administration of acamprosate prodrugs of Formula (I). The protocol is adapted from methods described in Yan et al., Neuropharmacology 2005, 49, 1053-66. Fmr1 KO mice aged 30-33 days receive vehicle or test article (at doses from 1 mg/kg to 200 mg/kg) by oral gavage (N=10 animals per dose group) 1 hour prior to testing for open-field activity. Mice are placed into the center of a clear Plexiglas (40×40×30 cm) open-field arena and allowed to explore for 30 minutes. Bright, overhead lighting provides approximately 800 lux of illumination inside the arenas. White noise is present at approximately 55 dB inside the arenas. Total distance traveled data during the minute test is collected in two-min intervals by a computer-operated Digiscan optical animal activity system (Accuscan Electronics), with data for the full 30-min test being analyzed. Open-field activity data is analyzed using a two-step process. First, the data from vehicle-treated WT and Fmr1 KO littermates are analyzed using a one-way ANOVA. Next, the Fmr1 KO data for three doses of each compound are analyzed to determine if the treatment significantly alters the behavior of the Fmr1 KO mice. There is a significant (p<0.001) increase in locomotor activity in vehicle-treated Fmr1 KO mice compared to vehicle-treated wild-type (WT) controls. In addition, certain compounds of Formula (I) produce a dose-related alteration in total distance traveled in Fmr1 KO mice in comparison to vehicle. Fmr1 KO mice that receive compounds of Formula (I) at a dose of 200 mg/kg are significantly less active than mice than vehicle-treated Fmr1 KO mice. These data show there is a dose related reduction in locomotor activity in Fmr1 KO mice treated with active compounds of Formula (I), indicating that these compounds reduce Fmr1 KO hyperactivity as assessed in this assay.

### Example 5

### Treatment of Fragile X Knockout Mice with Acamprosate Prodrugs in Prepulse Inhibition of Acoustic Startle

The goal of the experiment is to determine if prepulse inhibition of the acoustic startle response in Fmr1 KO mice is altered following administration of acamprosate prodrugs of Formula (I). The protocol is adapted from methods described in DeVrij, FMS; Neurobiol Dis. 2008, 127-132. Prepulse inhibition of startle (PPI) is measured by analysis of eye blink reactions of mice to acoustic stimuli, based on the magnetic distance measurement technique (MDMT) used for eye blink conditioning (Koekkoek et al., J. Neurophysiol. 2002, 88: 2124-33; Koekkoek et al., Neuron 2005, 47: 339-52). Adult Fmr1 KO mice (N=8) and wild type littermates (N=9) are anesthetized with an oxygenated mixture of nitrous oxide and isoflurane. A dental acrylic pedestal is placed on the skull and animals are allowed to recover for three days. Prior to the experiment, the mice are very briefly sedated using the isoflurane/nitrous oxide mixture. A sensor holder with an airchannel and a magnet sensor is attached to the pedestal. A small neobdimium iron borium magnet (0.8×1.6×0.2 mm) is glued to the lower eyelid with a minute drop of cyanoacrylate and a silicon body harness is put on to protect the mice from strain on the pedestal. Mice are placed inside their own cages within soundproof training chambers and allowed to recover until normal behavior (grooming, eating) returned, usually this is within 15 minutes. To test and calibrate the MDMT system, air puffs are given as a measure of full eyelid closure. A background noise level of 60 dB white noise is present. Subsequently, the mice are presented with a white noise startle stimulus of 90 dB, which in the prepulse inhibition condition is preceded by a 70 dB white noise prepulse, 50 ms before the startle stimulus. Each mouse is subjected to seven blocks of trials consisting of one air puff and three repeated measures of a startle stimulus followed fifty seconds later by a prepulse/startle stimulus with a fifty second intertrial interval. The next day the same mice are analyzed again in the same way after drug treatment. Animals receive vehicle or test article (at doses from 1 mg/kg to 200 mg/kg) by oral gavage. Active compounds of Formula (I) are those that significantly increase the percentage of PPI.

### Example 6

### Treatment of Fragile X Knockout Mice with Acamprosate Prodrugs in Marble-Burying Assay

The goal of the experiment is to determine if marble-burying behavior is reduced in Fmr1 KO mice following administration of acamprosate prodrugs of Formula (I). Fmr1 KO mice receive vehicle or test article (at doses from 1 mg/kg to 200 mg/kg) by oral gavage (N=10 animals per dose group) 1 hour prior to testing. A standard mouse cage is filled with 10 cm of corn-cob bedding. Twenty small (1.5-2 cm) black marbles are placed equidistant (about 1-2 cm apart) on top of the bedding. A mouse is placed in the cage and allowed to explore and bury the marbles. After about 20 minutes, the mouse is removed and the number of marbles buried (a marble is said to be “buried” if more than 50% of it is under the bedding) is recorded. Marbles buried are manually scored on a data sheet by an experimenter who is blind to the genotype and treatment. The data are then manually entered into a computer-spreadsheet and analyzed with a two-way (dose×treatment order) ANOVA. Significant main effects of dose are then analyzed using least squares follow-up comparisons. Administration of compounds of Formula (I) to fragile X knockout mice reduce marble burying behavior in a dose dependent manner. Active compounds of Formula (I) reduce the types of anxiety-like/obsessive/repetitive behaviors assessed in this assay.

### Example 7

### Hippocampal Neuronal Culture and Quantification of Protrusion Density and Dendrite Branching

The goal of the experiment is to determine the effect of acamprosate prodrugs of Formula (I) on dendrite morphology in hippocampal neurons isolated from Fmrl KO mice. The protocol is adapted from methods described in DeVrij, FMS; Neurobiol Dis. 2008, 127-132. E18 wild type and Fmr1 KO mice litters are planned on the same day. Embryos are decapitated after which hippocampi are removed and dissociated by trypsin and mechanical treatment. Neurons are plated on poly-L-lysine (100 μg/mL and laminin (50 μg/mL) coated 30 mm glass coverslips. The neurons are attached to the substrate in a drop of Neurobasal medium, containing penicillin/streptomycin, Glutamax and B-27 supplements (all from Gibco). After 2 hours, medium volume is adjusted to 2 mL per coverslip in 6-well plates. After 20 days in vitro cells are transfected, using Lipofectamine 2000, with an mCherry construct under control of a chicken β-actin promoter to ensure neuron-specific expression. One day after transfection, cells are either treated for 4 to 16 hours with various concentrations of compounds of Formula (I), or left untreated in supplemented Neurobasal medium. After treatment, neurons are fixed in 4% formaldehyde in PBS, washed in PBS and mounted in Mowiol mounting solution (Mowiol 4-88, Hoechst).

Images of β-actin-mCherry transfected neurons are acquired using a Zeiss LSM510 confocal microscope. Twenty to forty neurons from three independent experiments are imaged for each condition. For each neuron, a z-stack of 10×0.3 μm is made. The projected images are analyzed for protrusions with Metamorph software (Molecular Devices, Sunnyvale, Calif.). Two distal dendritic segments of 70-100 μm are chosen per neuron for protrusion morphometric analysis. For each protrusion, the length and the width are measured. The length is defined as the distance from the base to the tip of the protrusion; width is defined as the maximum distance perpendicular to the long axis of the protrusion. In order to make an objective distinction between spines and filopodia, the ratio of the width and the length is calculated for each protrusion. Protrusions with a ratio above or equal to 0.5 are considered as spines and conversely, protrusions with a ratio below 0.5 are considered as filopodia. Averages of protrusion densities of three independent experiments are compared with unpaired two-tailed Student's T-tests. Dendrite branching of/3-actin-mCherry transfected neurons is quantified by performing Sholl analyses of stacked Zeiss confocal generated images (40× objective, stack of 20×0.2 μm). With Metamorph software, concentric equally spaced circles (every 20 μm) are drawn around the cell soma of each neuron and subsequently, the amount of dendrite crossings are counted per circle. Averages of counts of three independent experiments are compared with unpaired two-tailed Student's T-tests. Active compounds of Formula (I) are those that cause a significant increase in mushroom protrusion and a decrease in filopodia.

### Example 8

### Treatment of Subjects with Fragile X Syndrome

A pharmaceutical composition comprising a compound of Formula (I) is administered orally to subjects with fragile X syndrome. These subjects have serious behavioral problems that are incompletely controlled with typical psychoactive medications. Doses are titrated from about 5 mg/kg/day to a maximum of about 50 mg/kg/day, with a duration of about 4 months. Clinicians rate their overall impression of improvement with treatment on a seven category scale ranging from “much worse,” “worse,” “slightly worse,” “no change,” “slightly better,” “better” or “much better”. Subjects are considered “Improved” if the clinician rating is either “much better” or “better”; considered “Not Improved” if the rating is “slightly worse”, “no change” or “slightly better”; and considered “Worsened” if rated “worse” or “much worse”. Subjects demonstrate an improvement in behavior, including less irritability, aggression and agitation. Other areas of improvement include increased class participation and decreased hyperactivity.

### Example 9

### Treatment of Subjects with Autism

A pharmaceutical composition comprising a compound of Formula (I) is administered orally to subjects with autism spectrum disorder. Doses are titrated from bout 5 mg/kg/day to a maximum of about 50 mg/kg/day, with a maximum duration of about 8 months. Improvements are noted in several cognitive and behavioral domains such as increased interest and response to spoken language and spontaneous attempts to communicate verbally. Dramatic improvements in mood and affect such as “looks comfortable, calm and happy” are also noted. Increased alertness, interest and motivation to work on cognitive/educational activities with school instructors are also noted. School personnel record behavior on a daily basis, and are not informed regarding changes in drug treatment for a given subject. Daily scores are averaged over the five weeks after initiating therapy and compared to the average scores for the five weeks immediately preceding initiation of therapy. Significant improvements following initiation of treatment with a compound of Formula (I) are noted in the following domains: episodes of social inappropriate behavior such as scratching, hitting and kicking others are found to decrease; episodes of self-abusive behavior such as hand biting or hitting of the head are found to decrease; episodes of eye diversion are found to decrease.

Finally, it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

What is claimed is:

1. A method of treating a subject, comprising administering to a subject having at least one condition selected from the group consisting of fragile X syndrome, fragile X-associated tremor/ataxia syndrome, Down's syndrome and autism, a pharmaceutical composition comprising an acamprosate prodrug.

2. The method of claim 1, wherein the acamprosate prodrug comprises a compound of Formula (I): 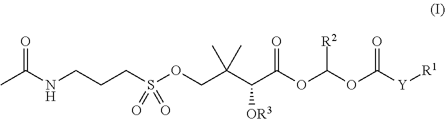
or a pharmaceutically acceptable salt thereof, wherein:
Y is chosen from —O— and a bond;
R¹ is chosen from C₁₋₆ alkyl, substituted C₁₋₆ alkyl, C₃₋₆ cycloalkyl, substituted C₃₋₆ cycloalkyl, phenyl, substituted phenyl, C₄₋₁₂ cycloalkylalkyl, substituted C₄₋₁₂ cycloalkylalkyl, C₇₋₁₂ arylalkyl, substituted C₇₋₁₂ arylalkyl, C₁₋₆ heteroalkyl, substituted C₁₋₆ heteroalkyl, C₃₋₆ heterocycloalkyl, substituted C₃₋₆ heterocycloalkyl, C₅₋₆ heteroaryl, substituted C₅₋₆ heteroaryl, C₄₋₁₂ heterocycloalkylalkyl, substituted C₄₋₁₂ heterocycloalkylalkyl, C₆₋₁₂ heteroarylalkyl, substituted C₆₋₁₂ heteroarylalkyl, and —(CHR⁶)ₙ—OPO(OH)₂ wherein n is chosen from 1, 2, and 3, and each R⁶ is independently chosen from hydrogen and methyl;
R² is chosen from hydrogen, C₁₋₆ alkyl, substituted C₁₋₆ alkyl, C₃₋₆ cycloalkyl, substituted C₃₋₆ cycloalkyl, phenyl, and substituted phenyl; and
R³ is chosen from hydrogen, —PO(OH)₂, and —C(O)R⁴ wherein R⁴ is C₁₋₄ alkyl.

3. The method of claim 2, wherein each substituent group is independently chosen from halogen, —OH, C₁₋₄ alkyl, C₁₋₄ alkoxy, and —N(R⁵)₂ wherein each R⁵ is independently chosen from hydrogen and C₁₋₂ alkyl.

4. The method of claim 2, wherein Y is —O—; and R¹ is chosen from C₁₋₄ alkyl, substituted C₁₋₄ alkyl, C₅₋₆ cycloalkyl, substituted C₅₋₆ cycloalkyl, phenyl, substituted phenyl, C₆₋₁₀ cycloalkylalkyl, substituted C₆₋₁₀ cycloalkylalkyl, C₇₋₁₀ arylalkyl, substituted C₇₋₁₀ arylalkyl, C₁₋₄ heteroalkyl, substituted C₁₋₄ heteroalkyl, C₅₋₆ heterocycloalkyl, substituted C₅₋₆ heterocycloalkyl, C₅₋₆ heteroaryl, substituted C₅₋₆ heteroaryl, C₆₋₁₀ heterocycloalkylalkyl, substituted C₆₋₁₀ heterocycloalkylalkyl, C₆₋₁₀ heteroarylalkyl, substituted C₆₋₁₀ heteroarylalkyl, and —(CHR⁶)ₙ—OPO(OH)₂, wherein n is chosen from 1, 2 and 3, and each R⁶ is independently chosen from hydrogen and methyl.

5. The method of claim 2, wherein Y is a bond; and R¹ is chosen from C₁₋₄ alkyl, substituted C₁₋₄ alkyl, C₅₋₆ cycloalkyl, substituted C₅₋₆ cycloalkyl, phenyl, substituted phenyl, C₆₋₁₀ cycloalkylalkyl, substituted C₆₋₁₀ cycloalkylalkyl, C₇₋₁₀ arylalkyl, substituted C₇₋₁₀ arylalkyl, C₁₋₄ heteroalkyl, substituted C₁₋₄ heteroalkyl, C₅₋₆ heterocycloalkyl, substituted C₅₋₆ heterocycloalkyl, C₅₋₆ heteroaryl, substituted C₅₋₆ heteroaryl, C₆₋₁₀ heterocycloalkylalkyl, substituted C₆₋₁₀ heterocycloalkylalkyl, C₆₋₁₀ heteroarylalkyl, substituted C₆₋₁₀ heteroarylalkyl, and —(CHR⁶)ₙ—OPO(OH)₂, wherein n is chosen from 1, 2 and 3, and each R⁶ is independently chosen from hydrogen and methyl.

6. The method of claim 2, wherein R¹ is chosen from methyl, ethyl, n-propyl, isopropyl, n-butyl, isobutyl, sec-butyl, tert-butyl, phenyl, o-tolyl, benzyl, cyclohexyl, and —(CHR⁶)ₙ—OPO(OH)₂, wherein n is chosen from 1, 2 and 3, and each R⁶ is independently chosen from hydrogen and methyl.

7. The method of claim 2, wherein R² is chosen from hydrogen, methyl, ethyl, n-propyl, and isopropyl.

8. The method of claim 2, wherein R³ is hydrogen.

9. The method of claim 2, wherein R³ is —PO(OH)₂.

10. The method of claim 2, wherein R³ is —C(O)R⁴ wherein R⁴ is C₁₋₄ alkyl.

11. The method of claim 2, wherein Y is —O—; R¹ is chosen from methyl, ethyl, n-propyl, isopropyl, n-butyl, isobutyl, sec-butyl, tert-butyl, phenyl, o-tolyl, benzyl, cyclohexyl, and —(CHR⁶)ₙ—OPO(OH)₂; R² is chosen from hydrogen, methyl, ethyl, n-propyl, isopropyl, cyclohexyl, and phenyl; and R³ is chosen from hydrogen and —PO(OH)₂.

12. The method of claim 2, wherein Y is a bond; R¹ is chosen from methyl, ethyl, n-propyl, isopropyl, n-butyl, isobutyl, sec-butyl, tert-butyl, phenyl, o-tolyl, benzyl, cyclohexyl, and —(CHR⁶)ₙ—OPO(OH)₂, wherein n is chosen from 1, 2 and 3; R² is chosen from hydrogen, methyl, ethyl, n-propyl, isopropyl, cyclohexyl, and phenyl; and R³ is chosen from hydrogen and —PO(OH)₂.

13. The method of claim 2, wherein the compound of Formula (I) is chosen from:
(methylethoxycarbonyloxy)methyl (2R)-4-{[3-(acetylamino)propyl]sulfonyloxy}-3,3-dimethyl-2-hydroxy-butanoate;
(2-phenylacetyloxy)methyl (2R)-4-{[3-(acetylamino)propyl]sulfonyloxy}-3,3-dimethyl-2-hydroxy-butanoate;
(2-methylpropanoyloxy)ethyl (2R)-4-{[3-(acetylamino)propyl]sulfonyloxy}-3,3-dimethyl-2-hydroxy-butanoate;
(2-methylpropanoyloxy)ethyl (2R)-4-{[3-(acetylamino)propyl]sulfonyloxy}-3,3-dimethyl-2-hydroxy-butanoate;
(1R)-(2-methylpropanoyloxy)ethyl (2R)-4-{[3-(acetylamino)propyl]sulfonyloxy}-3,3-dimethyl-2-hydroxy-butanoate;
ethoxycarbonyloxyethyl (2R)-4-{[3-(acetylamino)propyl]sulfonyloxy}-3,3-dimethyl-2-hydroxy-butanoate;
(1R)-1-ethoxycarbonyloxyethyl (2R)-4-{[3-(acetylamino)propyl]sulfonyloxy}-3,3-dimethyl-2-hydroxy-butanoate;
(1S)-1-ethoxycarbonyloxyethyl (2R)-4-{[3-(acetylamino)propyl]sulfonyloxy}-3,3-dimethyl-2-hydroxy-butanoate;
ethoxycarbonyloxyethyl (2R)-4-{[3-(acetylamino)propyl]sulfonyloxy}-3,3-dimethyl-2-(2-methylpropanoyloxy)-butanoate;
benzoyloxyethyl (2R)-4-{[3-(acetylamino)propyl]sulfonyloxy}-3,3-dimethyl-2-hydroxy-butanoate;
(methylethoxycarbonyloxy)ethyl (2R)-4-{[3-(acetylamino)propyl]sulfonyloxy}-2-hydroxy-3,3-dimethylbutanoate;
(1R)-(methylethoxycarbonyloxy)ethyl (2R)-4-{[3-(acetylamino)propyl]sulfonyloxy}-2-hydroxy-3,3-dimethylbutanoate;
(1S)-(methylethoxycarbonyloxy)ethyl (2R)-4-{[3-(acetylamino)propyl]sulfonyloxy}-2-hydroxy-3,3-dimethylbutanoate;
cyclohexyloxycarbonyloxyethyl (2R)-4-{[3-(acetylamino)propyl]sulfonyloxy}-2-hydroxy-3,3-dimethylbutanoate;
cyclohexylcarbonyloxyethyl (2R)-4-{[3-(acetylamino)propyl]sulfonyloxy}-2-hydroxy-3,3-dimethylbutanoate;
2-methyl-1-(methylethoxycarbonyloxy)propyl (2R)-4-{[3-(acetylamino)propyl]sulfonyloxy}-3,3-dimethyl-2-hydroxy-butanoate;
(2-methylpropanoyloxy)ethyl (2R)-4-{[3-(acetylamino)propyl]sulfonyloxy}-3,3-dimethyl-2-[(bishydroxyphosphinyl)oxy]butanoate;
cyclohexylcarbonyloxyethyl (2R)-4-{[3-(acetylamino)propyl]sulfonyloxy}-3,3-dimethyl-2-[(bishydroxyphosphinyl)oxy]butanoate;
ethoxycarbonyloxyethyl (2R)-4-{[3-(acetylamino)propyl]sulfonyloxy}-3,3-dimethyl-2-[(bishydroxyphosphinyl)oxy]butanoate;
(methylethoxycarbonyloxy)ethyl (2R)-4-{[3-(acetylamino)propyl]sulfonyloxy}-3,3-dimethyl-2-[(bishydroxyphosphinyl)oxy]butanoate; and
a pharmaceutically acceptable salt of any of the foregoing.

14. The method of claim 2, wherein the compound of Formula (I) is chosen from:
(2-methylpropanoyloxy)ethyl (2R)-4-{[3-(acetylamino)propyl]sulfonyloxy}-3,3-dimethyl-2-hydroxy-butanoate;
(1S)-(2-methylpropanoyloxy)ethyl (2R)-4-{[3-(acetylamino)propyl]sulfonyloxy}-3,3-dimethyl-2-hydroxy-butanoate;
(1R)-(2-methylpropanoyloxy)ethyl (2R)-4-{[3-(acetylamino)propyl]sulfonyloxy}-3,3-dimethyl-2-hydroxy-butanoate;
(methylethoxycarbonyloxy)ethyl (2R)-4-{[3-(acetylamino)propyl]sulfonyloxy}-2-hydroxy-3,3-dimethylbutanoate;
(1R)-(methylethoxycarbonyloxy)ethyl (2R)-4-{[3-(acetylamino)propyl]sulfonyloxy}-2-hydroxy-3,3-dimethylbutanoate;
(1S)-(methylethoxycarbonyloxy)ethyl (2R)-4-{[3-(acetylamino)propyl]sulfonyloxy}-2-hydroxy-3,3-dimethylbutanoate;
(2-methylpropanoyloxy)ethyl (2R)-4-{[3-(acetylamino)propyl]sulfonyloxy}-3,3-dimethyl-2-[(bishydroxyphosphinyl)oxy]butanoate;
(methylethoxycarbonyloxy)ethyl (2R)-4-{[3-(acetylamino)propyl]sulfonyloxy}-3,3-dimethyl-2-[(bishydroxyphosphinyl)oxy]butanoate; and
a pharmaceutically acceptable salt of any of the foregoing.

15. The method of claim 1, wherein the subject has fragile X syndrome.

16. The method of claim 1, wherein the subject has fragile X-associated tremor/ataxia syndrome.

17. The method of claim 1, wherein the subject has Down's syndrome.

18. The method of claim 1, wherein the subject has autism.

19. The method of claim 1, comprising administering to the subject at least one member selected from an mGluR antagonist, an antipsychotic agent, a GABA_{B} receptor agonist, a muscarinic receptor antagonist, a stimulant, a nicotinic receptor agonist, an endocannabinoid receptor antagonist, an AMPA agonist, an antidepressant, an α2-adrenergic agonist, and an anticonvulsant.
